(19) United States
(12) Patent Application Publication
(10) Pub. No.: US 2013/0231569 A1
(43) Pub. Date: Sep. 5, 2013

(54) MEDICAL ULTRASOUND 2-D TRANSDUCER ARRAY USING FRESNEL LENS APPROACH

(71) Applicants: KABUSHIKI KAISHA TOSHIBA, Tokyo (JP); TOSHIBA MEDICAL SYSTEMS CORPORATION, Otawara-Shi (JP)

(72) Inventor: Gregg MILLER, BOULDER, CO (US)

(73) Assignees: TOSHIBA MEDICAL SYSTEMS CORPORATION, Otawara-Shi (JP); KABUSHIKI KAISHA TOSHIBA, Tokyo (JP)

(21) Appl. No.: 13/865,124

(22) Filed: Apr. 17, 2013

Related U.S. Application Data
(62) Division of application No. 12/887,050, filed on Sep. 21, 2010, now abandoned.

(51) Int. Cl. A61B 8/00

(52) U.S. Cl. CPC A61B 8/4494; A61B 8/4444; A61B 8/4488
USPC 600/459

(57) ABSTRACT

The embodiments of the array include at least one first annular-like area and at least one second annular-like area that are concentric with each other. The second annular-like area substantially surrounds the first annular-like area. The first and second annular-like areas each exclusively include either dedicated transmit elements or dedicated receive elements. In addition, certain embodiments include a disabled third area or a spot of Argo located inside the first annular-like area and does not perform either transmit or receive function. In certain other embodiments, the first annular-like area and the third annular-like area are immediately juxtaposed without a gap. In yet other embodiments, the first annular-like area and the second annular-like area are immediately juxtaposed without a gap. Any of these areas are optionally dynamic and or steered.

Embodiments described herein relate generally to an ultrasound probe and method of operating the same.

### BACKGROUND

As illustrated in FIG. 20, a conventional ultrasound imaging system includes a processing unit 1, a display unit 2, a cable 3 and a transducer unit or ultrasound probe 4. The probe 4 is connected to the processing unit 1 via the cable 3. The processing unit 1 generally controls the transducer unit 4 for transmitting ultrasound pulses towards a region of interest in a patient and receiving the ultrasound echoes reflected from the patient. The processing unit 1 concurrently receives in real time the reflected ultrasound signals for further processing so as to display on the display unit 2 an image of the region of the interest.

In detail, the probe 4 further includes a predetermined number of transducer elements, which are grouped into channels for transmitting and receiving the ultrasound signals. For 2-dimensional (2D) imaging data, a number of elements ranges from 64 to 256. On the other hand, for 3-dimensional (3D) imaging data, a number of required channels often exceeds 1000's. In the above described conventional ultrasound imaging system, the probe 4 also houses a large number of electric components such as circuits and other components for controlling the transmission and reception of the ultrasound signals. In further detail, a transducer array of the probe includes the transducer array elements and the associated control circuitry to perform the generation of ultrasound pulses and the reception of the ultrasound echoes.

In general, the above described transducer array elements are shared transmit and receive elements that perform both transmit and receive functions within the same element. Because of the complex circuitries, the transducer arrays having the shared transmit and receive elements undesirably incur high costs and large power consumption among other things. To improve these disadvantages, prior art has attempted to separate the two functions in the transducer array elements. Although certain advantages have been gained by the dedicated transmit array elements and dedicated receive array elements, there remain some additional improvements to be made.

### BRIEF DESCRIPTION OF THE DRAWINGS

FIG. 1 is a diagram illustrating a first embodiment of the two-dimensional array in the probe according to the current invention.

FIG. 2 is a diagram illustrating a second embodiment of the two-dimensional array in the probe according to the current invention.

FIG. 3 is a diagram illustrating a third embodiment of the two-dimensional array in the probe according to the current invention.

FIG. 4 is a diagram illustrating a fourth embodiment of the two-dimensional array in the probe according to the current invention.

FIG. 5 is a diagram illustrating a fifth embodiment of the two-dimensional array in the probe according to the current invention.

FIG. 6 is a diagram illustrating a sixth embodiment of the two-dimensional array in the probe according to the current invention.

FIG. 7 is a diagram illustrating a seventh embodiment of the two-dimensional array in the probe according to the current invention.

FIG. 8 is a diagram illustrating an eighth embodiment of the two-dimensional array in the probe according to the current invention.

FIG. 9A is a diagram illustrating an embodiment of the array that is substantially the same as the first embodiment as illustrated in FIG. 1 and also illustrating a certain activation pattern or sequence of the dedicated receive elements in detecting the ultrasound echoes during the receiving operation.

FIG. 9B is a diagram illustrating an embodiment of the array that is substantially the same as the second embodiment as illustrated in FIG. 2 and also illustrating a certain activation pattern or sequence of the dedicated receive elements in detecting the ultrasound echoes during the receiving operation.

FIG. 9C is a diagram illustrating an embodiment of the array that is substantially the same as the third embodiment as illustrated in FIG. 3 and also illustrating a certain activation pattern or sequence of the dedicated receive elements in detecting the ultrasound echoes during the receiving operation.

FIG. 10A is a diagram illustrating an embodiment of the array that is substantially the same as the fourth embodiment as illustrated in FIG. 4 and also illustrating a certain activation pattern or sequence of the dedicated receive elements in detecting the ultrasound echoes during the receiving operation.

FIG. 10B is a diagram illustrating an embodiment of the array that is substantially the same as the fifth embodiment as illustrated in FIG. 5 and also illustrating a certain activation pattern or sequence of the dedicated receive elements in detecting the ultrasound echoes during the receiving operation.

FIG. 10C is a diagram illustrating an embodiment of the array that is substantially the same as the sixth embodiment as illustrated in FIG. 6 and also illustrating a certain activation pattern or sequence of the dedicated receive elements in detecting the ultrasound echoes during the receiving operation.

FIG. 11A is a diagram illustrating an embodiment of the array that is substantially the same as the fourth embodiment as illustrated in FIG. 4 and also illustrating another certain activation pattern or sequence of the dedicated receive elements and Spot of Arago and Spot of Arago in detecting the ultrasound echoes during the receiving operation.

FIG. 11B is a diagram illustrating an embodiment of the array that is substantially the same as the fifth embodiment as illustrated in FIG. 5 and also illustrating another certain activation pattern or sequence of the dedicated receive elements and Spot of Arago in detecting the ultrasound echoes during the receiving operation.

FIG. 11C is a diagram illustrating an embodiment of the array that is substantially the same as the sixth embodiment as illustrated in FIG. 6 and also illustrating another certain activation pattern or sequence of the dedicated receive elements in detecting the ultrasound echoes during the receiving operation.

FIG. 12A is a diagram illustrating an embodiment of the array that is substantially the same as a combination of the embodiments as illustrated in FIGS. 10A and 11A and also illustrates a certain first activation pattern or sequence of the dedicated receive elements and Spot of Arago in detecting the ultrasound echoes during the receiving operation.

FIG. 12B is a diagram illustrating a certain activation pattern or sequence of the dedicated receive elements of the same embodiment as described with respect to FIG. 12A in detecting the ultrasound echoes during the receiving operation and also illustrates a certain second activation pattern or sequence of the dedicated receive elements and Spot of Arago in detecting the ultrasound echoes during the receiving operation.

FIG. 12C is a diagram illustrating a certain activation pattern or sequence of the dedicated receive elements of the same embodiment as described with respect to FIG. 12A in detecting the ultrasound echoes during the receiving operation and also illustrates a certain third activation pattern or sequence of the dedicated receive elements and Spot of Arago in detecting the ultrasound echoes during the receiving operation.

FIG. 13 is a diagram illustrating an embodiment of the array in the probe that is substantially the same as the seventh embodiment as illustrated in FIG. 7 and also illustrates a certain activation pattern or sequence of the dedicated receive elements and Spot of Arago in detecting the ultrasound echoes during the receiving operation.

FIG. 14 is a diagram illustrating an embodiment of the array in the probe that is substantially the same as the eighth embodiment as illustrated in FIG. 8 and also illustrates a certain activation pattern or sequence of the dedicated receive elements and Spot of Arago in detecting the ultrasound echoes during the receiving operation.

FIGS. 15A, 15B and 15C are diagrams illustrating a spatial compounding aperture technique using an embodiment having the array in an elliptical arrangement.

FIGS. 16A, 16B and 16C are diagrams illustrating a synthetic aperture technique using an embodiment having the array in an elliptical arrangement.

FIGS. 17A and 17B are diagrams illustrating an asymmetric aperture technique using an embodiment having the array in an elliptical arrangement.

FIGS. 18A, 18B and 18C are diagrams illustrating another example of the asymmetric aperture technique using an embodiment having the array in an elliptical arrangement.

FIG. 19 is a diagram illustrating a ninth embodiment having non-overlapping annular-like areas according to the current invention.

FIG. 20 is a diagram illustrating one exemplary prior art ultrasound imaging system.

### DETAILED DESCRIPTION

Embodiments of the ultrasound transducer array according to the current invention include transducer elements that generate and transmit the ultrasound pulses towards a region of interest in a subject patient and receive the echoes reflected from the structures in the region of interest in the patient. The embodiments of the ultrasound transducer array according the current invention are two-dimensional arrays and generally include dedicated transmit elements and dedicated receive elements without shared transmit/receive elements. The embodiments of the ultrasound transducer array according the current invention are either sparsely or fully populated with the dedicated transmit elements and the dedicated receive elements. These transducer elements optionally include piezoelectric transducers, capacitive micromachined ultrasonic transducers (CMUTs), Piezoelectric micromachined ultrasonic transducers (pMUTs), or any other suitable type of transducers.

The dedicated transmit and receive elements are strictly separated and placed in a predetermined set of annular-like areas such as circular, elliptical and polygonal rings in the embodiments of the array according to the current invention. These embodiments of the array have several advantageous features according to the current invention. For example, the advantageous features include improvement in reduced electronic components associated with switching and electronic focusing, near field imaging performance due to large aperture, separation of transducer array element stackups for optimization of center frequency and bandwidth, and enhanced harmonic signal frequencies. Among the above advantages, the less electronic components also lead to desirable reduction in costs, power consumption and overall size. The separation of transducer array element stackups for transmit and receive may optimizes center frequency and bandwidth for each portion of the array through matching layer and/or PZT changes for the respective annular-like areas.

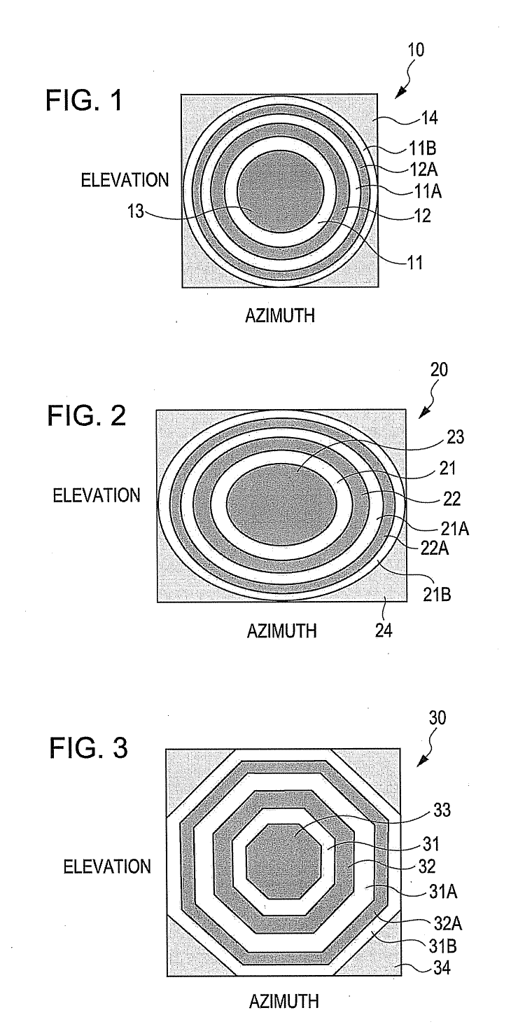

Referring now to the drawings, wherein like reference numerals designate corresponding structures throughout the views, and referring in particular to FIG. 1, a diagram illustrates an embodiment of the array in the probe according to the current invention. In general, the embodiment is a two-dimensional array 10 of transducer elements that includes dedicated transmit elements that perforin only transmit functions and dedicated receive elements that perform only receive functions. That is, the embodiment according to the current invention excludes any shared transmit/receive elements that perform both transmit and receive functions within the same element. The dedicated transmit elements and the dedicated receive elements are placed in a certain predetermined spatial arrangement as indicated by different shades of color in the diagram.

The dedicated transmit elements and the dedicated receive elements are both placed in annular-like circular areas including a first annular-like area 11 and a second annular-like area 12. As indicated by different shades, the first annular-like area 11 exclusively includes either one of dedicated transmit elements or dedicated receive elements while the second annular-like area 12 area exclusively includes the other one of the dedicated transmit elements and the dedicated receive elements. In other words, the first annular-like area 11 and the second annular-like area 12 alternate the dedicated transmit elements and the dedicated receive elements in their respective annular-like circular areas. For example, if the first annular-like area 11 exclusively includes the dedicated transmit elements, the second annular-like area 12 exclusively includes the dedicated receive elements. Furthermore, the second annular-like area 12 is immediately juxtaposed around the first annular-like area 11 and has a substantially concentric center with the first annular-like area 11.

As illustrated in the diagram, the first annular-like area 11 and the second annular-like area 12 are optionally repeated over a predetermined transducer surface of the two-dimensional array 10. As indicated by the shaded circular rings in the diagram, the additionally repeated annular-like areas 11A, 12A and 11B also exclusively have an alternate one of the dedicated transmit elements and the dedicated receive elements. In the illustrated embodiment, as the second annular-like area 12 is larger than the first annular-like area 11 and is immediately juxtaposed around the first annular-like area 11, the additionally repeated annular-like areas 11A, 12A and 11B also have substantially the same spatial relationship among them.

The term, “annular-like area” is intended to mean in the current patent application that each of the areas is delimited by a pair of substantially parallel outer and inner lines and or curves to form a contiguous strip of area surrounding a predetermined central portion or a donut-hole. Alternatively, the annular-like areas are also intended to mean in the current patent application that each of the areas is substantially concentric with each other while one of a pair of the annular-like areas is surrounded by the other adjacent larger one of the pair of the annular-like areas. Although the annular-like areas include circular and ecliptic rings, the annular-like areas are not limited to these specific shapes of rings and also include an optional combination of different shapes of the rings. For example, in case of polygonal rings, a pair of substantially concentric polygon edges defines each polygonal ring. The above examples do not limit the annular-like areas as used in the current patent application to particular shapes or sizes. Furthermore, the definition is for the spatial relation of the array elements and does not necessarily limit activation patterns or sequences of the dedicated transmit elements in transmitting the ultrasound pulses during the transmission operation. By the same token, the definition also does not necessarily limit activation patterns or sequences of the dedicated receive elements in detecting the ultrasound echoes during the receiving operation.

Still referring to FIG. 1, the exemplary embodiment additionally includes a third area 13 and a fourth area 14. The third area 13 is located inside the first annular-like area 11 and at least over the concentric center. The third area 13 is optionally juxtaposed to the first annular-like area 11 or alternatively contained in the first annular-like area 11 with a gap between the third area 13 and the first annular-like area 11. In this embodiment, as the third area 13 is indicated by the same shade as the second annular-like area 12, the third area 13 exclusively includes the same one of the dedicated transmit elements and the dedicated dedicated receive elements as the second annular-like area 12.

The above discussed repeated annular-like areas 11, 12 11A, 12A and 11B have a certain spatial relationship among them. To have a desired effect, these repeated annular-like areas 11, 12 11A, 12A and 11B switch from the dedicated transmit elements to the dedicated receive elements and vice versa at radii Rn as defined in the following equation:

$${r}_{n} = \sqrt{n λ f + \frac{{n}^{2} {λ}^{2}}{4}}$$

Where n is an integer while λ is the wavelength of the ultrasound wave the array is meant to focus and f is the distance from the center of the array to the focus. When the array is small compared to the focal length, this can be approximated by the following equation:

rₙ≅\sqrt{nλf}

For the arrays with many zones, the distance to the focus may be calculated if the radius of the outermost zone, r_{N} and its width Δ,r_{N}

$$f = \sqrt{\frac{2 {r}_{N} Δ {r}_{N}}{λ}}$$

In contrast, the fourth area 14 is located outside the largest annular-like area 11B on the two-dimensional array surface. The fourth area 14 is optionally void of any functional transducer element and/or disabled. Alternatively, the fourth area 14 is optionally populated by the dedicated transmit elements for maximum power or the dedicated receive elements for maximum sensitivity.

In one exemplary array, the embodiment includes a total of ten thousand (10,000) array elements with 100 Azimuth elements and 100 Elevation elements. Among the 10,000 array elements, assuming that predetermined numbers M and N respectively indicate a number of dedicated transmit elements and dedicated receive elements while a third number O indicates a number of array elements that is optionally unused, the sum of M+N+O is 10,000. For example, the first predetermined number M and the second predetermined number N are respectively 3750 dedicated transmit elements and 3750 dedicated receive elements while the third predetermined number O is 2500 unused array elements. Furthermore, based upon the above example, the 3750 dedicated transmit elements are optionally divided among the first annular-like areas 11, 11A and 11B whose area sizes are equal in one embodiment. In another embodiment, based upon the same example, the 3750 dedicated transmit elements are optionally divided among the first annular-like areas 11, 11A and 11B whose area sizes are not equal. By the same token, based upon the same example, the 3750 dedicated receive elements are optionally divided among the second annular-like areas 12 and 12A whose area sizes may or may not be equal. In an alternative embodiment, the third area 13 is optionally included in the second annular-like areas 12 and 12A for the purpose of populating the array elements.

In another exemplary embodiment, the array is optionally fully populated or sparsely populated by the dedicated transmit elements and the dedicated receive elements. In case of semi-sparsely populated rings, a predetermined Apodization function is applied to weight the detected signals for the purpose of shaping a beam profile.

FIG. 2 is a diagram illustrating a second embodiment of the array in the probe according to the current invention. In general, the embodiment is a two-dimensional array 20 of transducer elements that includes dedicated transmit elements that perform only transmit functions and dedicated receive elements that perform only receive functions. That is, the embodiment according to the current invention excludes any shared transmit/receive elements that perform both transmit and receive functions within the same element. The dedicated transmit elements and the dedicated receive elements are placed in a certain in a certain predetermined spatial arrangement as indicated by different shades of color in the diagram.

The dedicated transmit elements and the dedicated receive elements are both placed in annular-like elliptical areas including a first annular-like area 21 and a second annular-like area 22. As indicated by different shades, the first annular-like area 21 exclusively includes either one of dedicated transmit elements or dedicated receive elements while the second annular-like area 22 area exclusively includes the other one of the dedicated transmit elements and the dedicated receive elements. In other words, the first annular-like area 21 and the second annular-like area 22 alternate the dedicated transmit elements and the dedicated receive elements in their respective annular-like elliptical areas. For example, if the first annular-like area 21 exclusively includes the dedicated transmit elements, the second annular-like area 22 exclusively includes the dedicated receive elements. Furthermore, the second annular-like area 22 is immediately juxtaposed around the first annular-like area 21 and has a substantially concentric center with the first annular-like area 21.

As illustrated in the diagram, the first annular-like area 21 and the second annular-like area 22 are optionally repeated over a predetermined transducer surface of the two-dimensional array 20. As indicated by the shaded elliptical rings in the diagram, the additionally repeated annular-like areas 21A, 22A and 21B also exclusively have an alternate one of the dedicated transmit elements and the dedicated receive elements. In the illustrated embodiment, as the second annular-like area 22 is larger than the first annular-like area 21 and is immediately juxtaposed around the first annular-like area 21, the additionally repeated annular-like areas 21A, 22A and 21B also have substantially the same spatial relationship among them. The term, “annular-like area” is intended to have the same meaning as already described with respect to FIG. 1 in the in the current patent application.

Still referring to FIG. 2, the exemplary embodiment additionally includes a third area 23 and a fourth area 24. The third area 23 is located inside the first annular-like area 21 and at least over the concentric center. The third area 23 is optionally juxtaposed to the first annular-like area 21 or alternatively contained in the first annular-like area 21 with a gap between the third area 23 and the first annular-like area 21. In this embodiment, as the third area 23 is indicated by the same shade as the second annular-like area 22, the third area 23 exclusively includes the same one of the dedicated transmit elements and the dedicated receive elements as the second annular-like area 22. In contrast, the fourth area 24 is located outside the largest annular-like area 21B on the two-dimensional array surface. The fourth area 24 is optionally void of any functional transducer element and/or disabled. Alternatively, the fourth area 24 is optionally populated by the dedicated transmit elements for maximum power or the dedicated receive elements for maximum sensitivity.

In one exemplary array, the embodiment includes a total of ten thousand (10,000) array elements with 100 Azimuth elements and 100 Elevation elements. Among the 10,000 array elements, assuming that predetermined numbers M and N respectively indicate a number of dedicated transmit elements and dedicated receive elements while a third number O indicates a number of array elements that is optionally unused, the sum of M+N+O is 10,000. For example, the first predetermined number M and the second predetermined number N are respectively 3750 dedicated transmit elements and 3750 dedicated receive elements while the third predetermined number O is 2500 unused array elements. Furthermore, based upon the above example, the 3750 dedicated transmit elements are optionally divided among the first annular-like areas 21, 21A and 21B whose area sizes are equal in one embodiment. In another embodiment, based upon the same example, the 3750 dedicated transmit elements are optionally divided among the first annular-like areas 21, 21A and 21B whose area sizes are not equal. By the same token, based upon the same example, the 3750 dedicated receive elements are optionally divided among the second annular-like areas 22 and 22A whose area sizes may or may not be equal. In an alternative embodiment, the third area 23 is optionally included in the second annular-like areas 22 and 22A for the purpose of populating the array elements.

In another exemplary embodiment, the array is optionally fully populated or sparsely populated by the dedicated transmit elements and the dedicated receive elements. In case of semi-sparsely populated rings, a predetermined Apodization function is applied to weight the detected signals for the purpose of shaping a beam profile.

FIG. 3 is a diagram illustrating a third embodiment of the array in the probe according to the current invention. In general, the embodiment is a two-dimensional array 30 of transducer elements that includes dedicated transmit elements that perform only transmit functions and dedicated receive elements that perform only receive functions. That is, the embodiment according to the current invention excludes any shared transmit/receive elements that perform both transmit and receive functions within the same element. The dedicated transmit elements and the dedicated receive elements are placed in a certain predetermined spatial arrangement as indicated by different shades of color in the diagram.

The dedicated transmit elements and the dedicated receive elements are both placed in annular-like polygonal areas including a first annular-like area 31 and a second annular-like area 32. As indicated by different shades, the first annular-like area 31 exclusively includes either one of dedicated transmit elements or dedicated receive elements while the second annular-like area 32 area exclusively includes the other one of the dedicated transmit elements and the dedicated receive elements. In other words, the first annular-like area 31 and the second annular-like area 32 alternate the dedicated transmit elements and the dedicated receive elements in their respective annular-like polygonal areas. For example, if the first annular-like area 31 exclusively includes the dedicated transmit elements, the second annular-like area 32 exclusively includes the dedicated receive elements. Furthermore, the second annular-like area 32 is immediately juxtaposed around the first annular-like area 31 and has a substantially concentric center with the first annular-like area 31.

As illustrated in the diagram, the first annular-like area 31 and the second annular-annular-like area 32 are optionally repeated over a predetermined transducer surface of the two-dimensional array 30. As indicated by the shaded polygonal rings in the diagram, the additionally repeated annular-like areas 31A, 32A and 31B also exclusively have an alternate one of the dedicated transmit elements and the dedicated receive elements. In the illustrated embodiment, as the second annular-like area 32 is larger than the first annular-like area 31 and is immediately juxtaposed around the first annular-like area 31, the additionally repeated annular-like areas 31A, 32A and 31B also have substantially the same spatial relationship among them. The term, “annular-like area” is intended to have the same meaning as already described with respect to FIG. 1 in the in the current patent application.

Still referring to FIG. 3, the exemplary embodiment additionally includes a third area 33 and a fourth area 34. The third area 33 is located inside the first annular-like area 31 and at least over the concentric center. The third area 33 is optionally juxtaposed to the first annular-like area 31 or alternatively contained in the first annular-like area 31 with a gap between the third area 33 and the first annular-like area 31. In this embodiment, as the third area 33 is indicated by the same shade as the second annular-like area 32, the third area 33 exclusively includes the same one of the dedicated transmit elements and the dedicated receive elements as the second annular-like area 32.

In contrast, the fourth area 34 is located outside the largest annular-like area 31B on the two-dimensional array surface. The fourth area 34 is optionally void of any functional transducer element or disabled. Alternatively, the fourth area 34 is optionally populated by the dedicated transmit elements for maximum power or the dedicated receive elements for maximum sensitivity.

In one exemplary array, the embodiment includes a total of ten thousand (10,000) array elements with 100 Azimuth elements and 100 Elevation elements. Among the 10,000 array elements, assuming that predetermined numbers M and N respectively indicate a number of dedicated transmit elements and dedicated receive elements while a third number third number O indicates a number of array elements that is optionally unused, the sum of M+N+O is 10,000. For example, the first predetermined number M and the second predetermined number N are respectively 3750 dedicated transmit elements and 3750 dedicated receive elements while the third predetermined number O is 2500 unused array elements. Furthermore, based upon the above example, the 3750 dedicated transmit elements are optionally divided among the first annular-like areas 31, 31A and 31B whose area sizes are equal in one embodiment. In another embodiment, based upon the same example, the 3750 dedicated transmit elements are optionally divided among the first annular-like areas 31, 31A and 31B whose area sizes are not equal. By the same token, based upon the same example, the 3750 dedicated receive elements are optionally divided among the second annular-like areas 32 and 32A whose area sizes may or may not be equal. In an alternative embodiment, the third area 33 is optionally included in the second annular-like areas 32 and 32A for the purpose of populating the array elements.

In another exemplary embodiment, the array is optionally fully populated or sparsely populated by the dedicated transmit elements and the dedicated receive elements. In case of semi-sparsely populated rings, a predetermined Apodization function is applied to weight the detected signals for the purpose of shaping a beam profile.

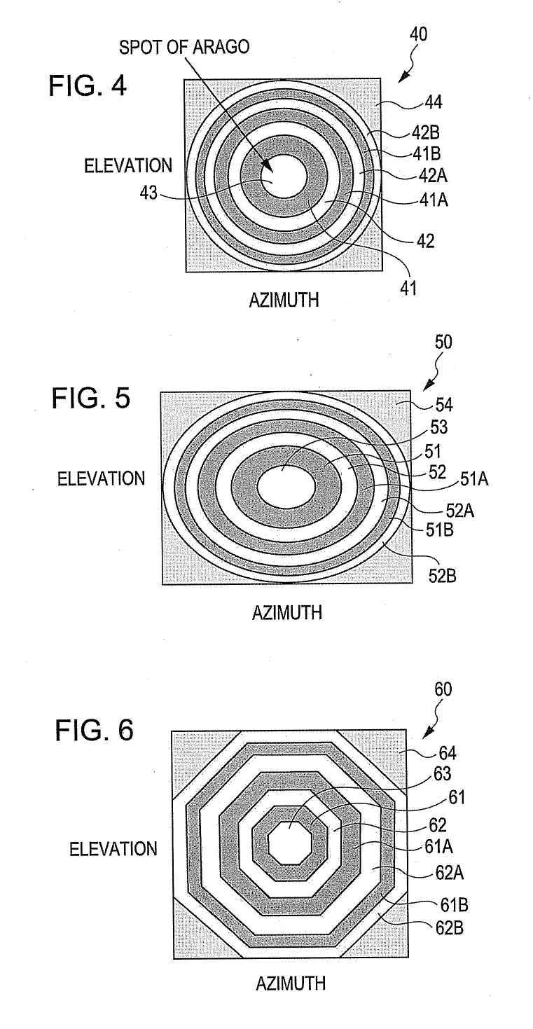

FIG. 4 is a diagram illustrating a fourth embodiment of the array in the probe according to the current invention. In general, the embodiment is a two-dimensional array 40 of transducer elements that includes dedicated transmit elements that perform only transmit functions and dedicated receive elements that perform only receive functions. That is, the embodiment according to the current invention excludes any shared transmit/receive elements that perform both transmit and receive functions within the same element. The dedicated transmit elements and the dedicated receive elements are placed in a certain predetermined spatial arrangement as indicated by different shades of color in the diagram.

The dedicated transmit elements and the dedicated receive elements are both placed in annular-like circular areas including a first annular-like area 41 and a second annular-like area 42. As indicated by different shades, the first annular-like area 41 exclusively includes either one of dedicated transmit elements or dedicated receive elements while the second annular-like area 42 area exclusively includes the other one of the dedicated transmit elements and the dedicated receive elements. In other words, the first annular-like area 41 and the second annular-like area 42 alternate the dedicated transmit elements and the dedicated receive elements in their respective annular-like circular areas. For example, if the first annular-like area 41 exclusively includes the dedicated transmit elements, the second annular-like area 42 exclusively includes the dedicated receive elements. Furthermore, the second annular-like area 42 is immediately juxtaposed around the first annular-like area 41 and has a substantially concentric center with the first annular-like area 41.

As illustrated in the diagram, the first annular-like area 41 and the second annular-like area 42 are optionally repeated over a predetermined transducer surface of the two-dimensional array 40. As indicated by the shaded circular rings in the diagram, the additionally repeated annular-like areas 41A, 42A, 41B and 42B also exclusively have an alternate one of the dedicated transmit elements and the dedicated receive elements. In the illustrated embodiment, as the second annular-like area 42 is larger than the first annular-like area 41 and is immediately juxtaposed around the first annular-like area 41, the additionally repeated annular-like areas 41A, 42A, 41B and 42B also have substantially the same spatial relationship among them. The term, “annular-like area” is intended to have the same meaning as already described with respect to FIG. 1 in the in the current patent application.

Still referring to FIG. 4, the exemplary embodiment additionally includes a third area 43 and/or a fourth area 44. The third area 43 is a circle and is located inside the first annular-like area 41 and at least over the concentric center. The third area 43 is optionally juxtaposed to the first annular-like area 41 or alternatively contained in the first annular-like area 41 with a gap between the third area 43 and the first annular-like area 41. In this embodiment, the third area 43 is indicated in white that the third area 43 is devoid of the dedicated transmit elements and the dedicated receive elements or is alternatively disabled. The third area 43 optionally further reduces the number of array elements and ultimately improves the electronic circuitry cost, the power consumption and the size. The third area 43 also results in improved beam width thereby enhancing near-field lateral resolution in improving imaging quality. Since the third area 43 having non-functioning array elements or lacking array elements correlates with the opaque optical disk in a first Fresnel zone which produces the spot of Arago in optics diffraction theory, the third area 43 is also called Spot of Arago in the =Tent application.

The above discussed repeated annular-like areas 41, 42 41A, 42A, 41B and 42B have a certain spatial relationship among them. To have a desired effect, these repeated annular-like areas 41, 42 41A, 42A, 41B and 42B switch from the dedicated transmit elements to the dedicated receive elements and vice versa at radii Rn as defined in the following equation:

$${r}_{n} = \sqrt{n λ f + \frac{{n}^{2} {λ}^{2}}{4}}$$

Where n is an integer while λ is the wavelength of the ultrasound wave the array is meant to focus and f is the distance from the center of the array to the focus. When the array is small compared to the focal length, this can be approximated by the following equation:

rₙ≅\sqrt{nλf}

For the arrays with many zones, the distance to the focus may be calculated if the radius of the outermost zone, r_{N} and its width Δ,r_{N}

$$f = \sqrt{\frac{2 {r}_{N} Δ {r}_{N}}{λ}}$$

In contrast, the fourth area 44 is located outside the largest annular-like area 42B on the two-dimensional array surface. The fourth area 44 is optionally disabled or devoid of any functional transducer element. Alternatively, the fourth area 44 is optionally populated by the dedicated transmit elements for maximum power and/or the dedicated receive elements for maximum sensitivity.

In one exemplary array, the embodiment includes a total of ten thousand (10,000) array elements with 100 Azimuth elements and 100 Elevation elements. Among the 10,000 array elements, assuming that predetermined numbers M and N respectively indicate a number of dedicated transmit elements and dedicated receive elements while a third number O indicates a number of array elements that is unused, the sum of M+N+O is 10,000. For example, the first predetermined number M and the second predetermined number N are respectively 3750 dedicated transmit elements and 3750 dedicated receive elements while the third predetermined number O is 2500 unused array elements. Furthermore, based upon the above example, the 3750 dedicated transmit elements are optionally divided among the first annular-like areas 41, 41A and 41B whose area sizes are equal in one embodiment. In another embodiment, based upon the same example, the 3750 dedicated transmit elements are optionally divided among the first annular-like areas 41, 41A and 41B whose area sizes are not equal. By the same token, based upon the same example, the 3750 dedicated receive elements are optionally divided among the second annular-like areas 42, 42A and 42B whose area sizes may or may not be equal. In an alternative embodiment, the third area 43 is included in the number N if the third area 43 is equipped with array elements and unused.

In another exemplary embodiment, the array is optionally fully populated or sparsely populated by the dedicated transmit elements and the dedicated receive elements. In case of semi-sparsely populated rings, a predetermined Apodization function is applied to weight the detected signals for the purpose of shaping a beam profile.

FIG. 5 is a diagram illustrating a fifth embodiment of the array in the probe according to the current invention. In general, the embodiment is a two-dimensional array 50 of transducer elements that includes dedicated transmit elements that perform only transmit functions and dedicated receive elements that perform only receive functions. That is, the embodiment according to the current invention excludes any shared transmit/receive elements that perform both transmit and receive functions within the same element. The dedicated transmit elements and the dedicated receive elements are placed in a certain predetermined spatial arrangement as indicated by different shades of color in the diagram.

The dedicated transmit elements and the dedicated receive elements are both placed in annular-like elliptical areas including a first annular-like area 51 and a second annular-like area 52. As indicated by different shades, the first annular-like area 51 exclusively includes either one of dedicated transmit elements or dedicated receive elements while the second annular-like area 52 area exclusively includes the other one of the dedicated transmit elements and the dedicated receive elements. In other words, the first annular-like area 51 and the second annular-like area 52 alternate the dedicated transmit elements and the dedicated receive elements in their respective annular-like elliptical areas. For example, if the first annular-like area 51 exclusively includes the dedicated transmit elements, the second annular-like area 52 exclusively includes the dedicated receive elements. Furthermore, the second annular-like area 52 is immediately juxtaposed around the first annular-like area 51 and has a substantially concentric center with the first annular-like area 51.

As illustrated in the diagram, the first annular-like area 51 and the second annular-like area 52 are optionally repeated over a predetermined transducer surface of the two-dimensional array 50. As indicated by the shaded elliptical rings in the diagram, the additionally repeated annular-like areas 51A, 52A, 51B and 52B also exclusively have an alternate one of the dedicated transmit elements and the dedicated receive elements. In the illustrated embodiment, as the second annular-like area 52 is larger than the first annular-like area 51 and is immediately juxtaposed around the first annular-like area 51, the additionally repeated annular-like areas 51A, 52A, 51B and 52B also have substantially the same spatial relationship among them. The term, “annular-like area” is intended to have the same meaning as already described with respect to FIG. 1 in the in the current patent application.

Still referring to FIG. 5, the exemplary embodiment additionally includes a third area 53 and/or a fourth area 54. The third area 53 is an ellipse and is located inside the first annular-like area 51 and at least over the concentric center. The third area 53 is optionally juxtaposed to the first annular-like area 51 or alternatively contained in the first annular-like area 51 with a gap between the third area 53 and the first annular-like area 51. In this embodiment, the third area 53 is indicated in white that the third area 53 is devoid of the dedicated transmit elements and the dedicated receive elements or is alternatively disabled. The third area 53 optionally further reduces the number of array elements and ultimately improves the cost, the power consumption and the size. The third area 53 also results in improved beam width and thereby enhances near-field lateral resolution in improving imaging quality. Since the third area 53 having non-functioning array elements or lacking array elements correlates with the opaque optical disk in a first Fresnel zone which produces the spot of Arago in optics diffraction theory, the third area 53 is also called Spot of Arago in the current application.

In contrast, the fourth area 54 is located outside the largest annular-like area 52B on the two-dimensional array surface. The fourth area 54 is optionally disabled or devoid of any functional transducer element. Alternatively, the fourth area 54 is optionally populated by the dedicated transmit elements for maximum power and/or the dedicated receive elements for maximum sensitivity.

In one exemplary array, the embodiment includes a total of ten thousand (10,000) array elements with 100 Azimuth elements and 100 Elevation elements. Among the 10,000 array elements, assuming that predetermined numbers M and N respectively indicate a indicate a number of dedicated transmit elements and dedicated receive elements while a third number O indicates a number of array elements that is unused, the sum of M+N+O is 10,000. For example, the first predetermined number M and the second predetermined number N are respectively 3750 dedicated transmit elements and 3750 dedicated receive elements while the third predetermined number O is 2500 unused array elements. Furthermore, based upon the above example, the 3750 dedicated transmit elements are optionally divided among the first annular-like areas 51, 51A and 51B whose area sizes are equal in one embodiment. In another embodiment, based upon the same example, the 3750 dedicated transmit elements are optionally divided among the first annular-like areas 51, 51A and 51B whose area sizes are not equal. By the same token, based upon the same example, the 3750 dedicated receive elements are optionally divided among the second annular-like areas 52, 52A and 52B whose area sizes may or may not be equal. In an alternative embodiment, the third area 53 is included in the number N if the third area 53 is equipped with array elements and unused.

In another exemplary embodiment, the array is optionally fully populated or sparsely populated by the dedicated transmit elements and the dedicated receive elements. In case of semi-sparsely populated rings, a predetermined Apodization function is applied to weight the detected signals for the purpose of shaping a beam profile.

FIG. 6 is a diagram illustrating a sixth embodiment of the array in the probe according to the current invention. In general, the embodiment is a two-dimensional array 60 of transducer elements that includes dedicated transmit elements that perform only transmit functions and dedicated receive elements that perform only receive functions. That is, the embodiment according to the current invention excludes any shared transmit/receive elements that perform both transmit and receive functions within the same element. The dedicated transmit elements and the dedicated receive elements are placed in a certain predetermined spatial arrangement as indicated by different shades of color in the diagram.

The dedicated transmit elements and the dedicated receive elements are both placed in annular-like polygonal areas including a first annular-like area 61 and a second annular-like area 62. As indicated by different shades, the first annular-like area 61 exclusively includes either one of dedicated transmit elements or dedicated receive elements while the second annular-like area 62 area exclusively includes the other one of the dedicated transmit elements and the dedicated receive elements. In other words, the first annular-like area 61 and the second annular-like area 62 alternate the dedicated transmit elements and the dedicated receive elements in their respective annular-like polygonal areas. For example, if the first annular-like area 61 exclusively includes the dedicated transmit elements, the second annular-like area 62 exclusively includes the dedicated receive elements. Furthermore, the second annular-like area 62 is immediately juxtaposed around the first annular-like area 61 and has a substantially concentric center with the first annular-like area 61.

As illustrated in the diagram, the first annular-like area 61 and the second annular-like area 62 are optionally repeated over a predetermined transducer surface of the two-dimensional array 60. As indicated by the shaded polygonal rings in the diagram, the additionally repeated annular-like areas 61A, 62A, 61B and 62B also exclusively have an alternate one of the dedicated transmit elements and the dedicated receive elements. In the illustrated embodiment, as the second annular-like area 62 is larger than the first annular-like area 61 and is immediately juxtaposed around the first annular-like area 61, the additionally repeated annular-like areas 61A, 62A, 61B and 62B also have substantially the same spatial relationship among them. The term, “annular-like area” is intended to have the same meaning as already described with respect to FIG. 1 in the in the current patent application.

Still referring to FIG. 6, the exemplary embodiment additionally includes a third area 63 and/or a fourth area 64. The third area 63 is a polygon and is located inside the first annular-like area 61 and at least over the concentric center. The third area 63 is optionally juxtaposed to the first annular-like area 61 or alternatively contained in the first annular-like area 61 with a gap between the third area 63 and the first annular-like area 61. In this embodiment, the third area 63 is indicated in white that the third area 63 is devoid of the dedicated transmit elements and the dedicated receive elements or is alternatively disabled. The third area 63 optionally further reduces the number of array elements and ultimately improves the cost, the power consumption and the size. The third area 63 also results in improved beam width and thereby enhances near-field lateral resolution in improving imaging quality. Since the third area 63 having non-functioning array elements or lacking array elements correlates with the opaque optical disk in a first Fresnel zone which produces the spot of Arago in optics diffraction theory, the third area 63 is also called Spot of Arago in the current application.

In contrast, the fourth area 64 is located outside the largest annular-like area 62B on the two-dimensional array surface. The fourth area 64 is optionally disabled and/or devoid of any functional transducer element. Alternatively, the fourth area 64 is optionally populated by the dedicated transmit elements for maximum power and/or the dedicated receive elements for maximum sensitivity.

In one exemplary array, the embodiment includes a total of ten thousand (10,000) array elements with 100 Azimuth elements and 100 Elevation elements. Among the 10,000 array elements, assuming that predetermined numbers M and N respectively indicate a number of dedicated transmit elements and dedicated receive elements while a third number O indicates a number of array elements that is unused, the sum of M+N+O is 10,000. For example, the first predetermined number M and the second predetermined number N are respectively 3750 dedicated transmit elements and 3750 dedicated receive elements while the third predetermined number O is 2500 unused array elements. Furthermore, based upon the above example, the 3750 dedicated transmit elements are optionally divided among the first annular-like areas 61, 61A and 61B whose area sizes are equal in one embodiment. In another embodiment, based upon the same example, the 3750 dedicated transmit elements are optionally divided among the first annular-like areas 61, 61A and 61B whose area sizes are not equal. By the same token, based upon the same example, the 3750 dedicated receive elements are optionally divided among the second annular-like areas 62, 62A and 62B whose area sizes may or may not be equal. In an alternative embodiment, the third area 63 alternative embodiment, the third area 63 is included in the number N if the third area 63 is equipped with array elements and unused.

In another exemplary embodiment, the array is optionally fully populated or sparsely populated by the dedicated transmit elements and the dedicated receive elements. In case of semi-sparsely populated rings, a predetermined Apodization function is applied to weight the detected signals for the purpose of shaping a beam profile.

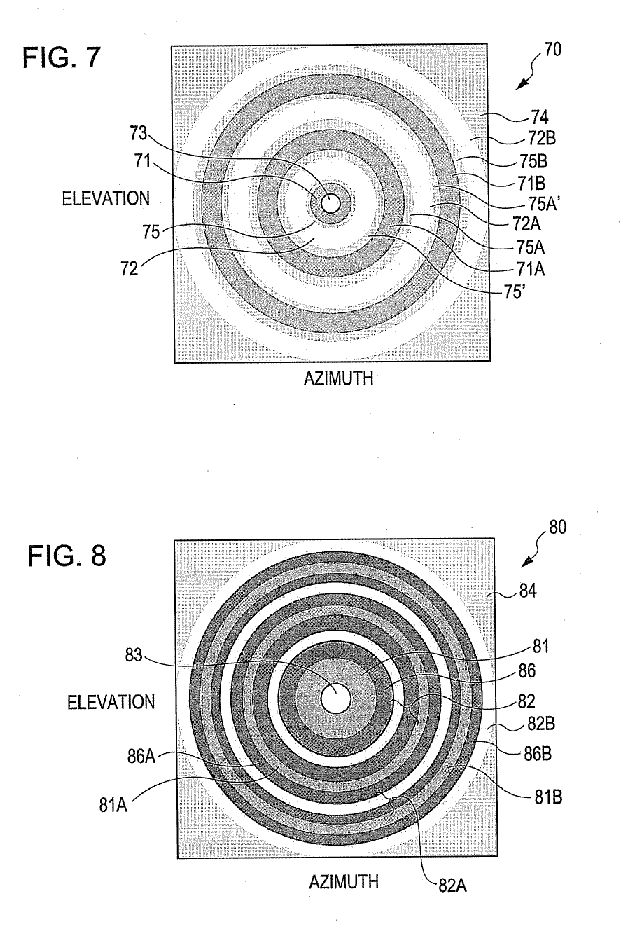

FIG. 7 is a diagram illustrating a seventh embodiment of the array in the probe according to the current invention. In general, the embodiment is a two-dimensional array 70 of transducer elements that includes dedicated transmit elements that perform only transmit functions and dedicated receive elements that perform only receive functions. That is, the embodiment according to the current invention excludes any shared transmit/receive elements that perform both transmit and receive functions within the same element. The dedicated transmit elements and the dedicated receive elements are placed in a certain predetermined spatial arrangement as indicated by different shades of color in the diagram.

The dedicated transmit elements and the dedicated receive elements are both placed in annular-like circular areas including a first annular-like area 71 and a second annular-like area 72. As indicated by different shades, the first annular-like area 71 exclusively includes either one of dedicated transmit elements or dedicated receive elements while the second annular-like area 72 area exclusively includes the other one of the dedicated transmit elements and the dedicated receive elements. In other words, the first annular-like area 71 and the second annular-like area 72 alternate the dedicated transmit elements and the dedicated receive elements in their respective annular-like circular areas. For example, if the first annular-like area 71 exclusively includes the dedicated transmit elements, the second annular-like area 72 exclusively includes the dedicated receive elements. Although the second annular-like area 72 is not immediately juxtaposed around the first annular-like area 71, the second annular-like area 72 has a substantially concentric substantially concentric center with the first annular-like area 71.

In the seventh embodiment of the array in the probe, there is an optional annular-like area 75 between the first annular-like area 71 and the second annular-like area 72. The optional annular-like area 75 is optionally populated with either one of the dedicated transmit elements or the dedicated receive elements, and these elements may be also optionally used or disabled. Alternatively, the optional annular-like area 75 is optionally populated with neither one of the dedicated transmit elements or the dedicated receive elements. Furthermore, an additional optional annular-like area 75′ surrounds the second annular-like area 72, and the additional optional annular-like area 75′ may be implemented in a similar manner as the optional annular-like area 75.

As illustrated in the diagram, the first annular-like area 71 and the second annular-like area 72 are optionally repeated over a predetermined transducer surface of the two-dimensional array 70. As indicated by the shaded circular rings in the diagram, the additionally repeated annular-like areas 71A, 72A, 71B and 72B also exclusively have an alternate one of the dedicated transmit elements and the dedicated receive elements. In the illustrated embodiment, as the second annular-like area 72 is larger than the first annular-like area 71 and is not immediately juxtaposed around the first annular-like area 71, the additionally repeated annular-like areas 71A, 72A, 71B and 72B also have substantially the same spatial relationship among them. By the same token, the additionally repeated annular-like areas 71A, 72A, 71B and 72B are interlaced by optional annular-like areas 75A and 75B as well as by additional optional annular-like area 75A′. The term, “annular-like area” is intended to have the same meaning as already described with respect to FIG. 1 in the in the current patent application.

Still referring to FIG. 7, the exemplary embodiment additionally includes a third area 73 and a fourth area 74. The third area 73 is a circle and is located inside the first annular-like area 71 and at least over the concentric center. The third area 73 is optionally juxtaposed to the first annular-like area 71 or alternatively contained in the first annular-like annular-like area 71 with a gap between the third area 73 and the first annular-like area 71. In this embodiment, the third area 73 is indicated in white that the third area 73 is devoid of the dedicated transmit elements and the dedicated receive elements or is alternatively disabled. The third area 73 optionally further reduces the number of array elements and ultimately improves the cost, the power consumption and the size. The third area 73 also results in improved beam width and thereby enhances near-field lateral resolution in improving imaging quality. Since the third area 73 having non-functioning array elements or lacking array elements correlates with the opaque optical disk in a first Fresnel zone which produces the spot of Arago in optics diffraction theory, the third area 73 is also called Spot of Arago in the current application.

In contrast, the fourth area 74 is located outside the largest annular-like area 72B on the two-dimensional array surface. The fourth area 74 is optionally disabled or devoid of any functional transducer element. Alternatively, the fourth area 74 is optionally populated by the dedicated transmit elements for maximum power or the dedicated receive elements for maximum sensitivity.

In one exemplary array, the embodiment includes a total of ten thousand (10,000) array elements with 100 Azimuth elements and 100 Elevation elements. Among the 10,000 array elements, assuming that predetermined numbers M and N respectively indicate a number of dedicated transmit elements and dedicated receive elements while a third number O indicates a number of array elements that is unused, the sum of M+N+O is 10,000. For example, the first predetermined number M and the second predetermined number N are respectively 3750 dedicated transmit elements and 3750 dedicated receive elements while the third predetermined number O is 2600 unused array elements. Furthermore, based upon the above example, the 3750 dedicated transmit elements are optionally divided among the first annular-like areas 71, 71A and 71B whose area sizes are equal in one embodiment. In another embodiment, based upon the same example, the 3750 dedicated transmit elements are optionally divided among the first annular-like areas 71, 71A and 71B whose area sizes are not equal. By the same token, based upon the same example, the 3750 dedicated receive elements are optionally divided among the second annular-like areas 72, 72A and 72B annular-like areas 72, 72A and 72B whose area sizes may or may not be equal. In an alternative embodiment, the third area 73 is included in the number N if the third area 73 is equipped with array elements and unused.

In another exemplary embodiment, the array is optionally fully populated or sparsely populated by the dedicated transmit elements and the dedicated receive elements. In case of semi-sparsely populated rings, a predetermined Apodization function is applied to weight the detected signals for the purpose of shaping a beam profile.

In addition to the above illustrated embodiment, alternative embodiments based upon the seventh embodiment further include an elliptical embodiment and a polygonal embodiment. In the elliptical alternative embodiment, the dedicated transmit elements and the dedicated receive elements are both placed in annular-like elliptical areas including a first annular-like area and a second annular-like area as described with respect to the seventh embodiment. Similarly, the third area, the fourth and the fifth area also exist in the elliptical alternative embodiment in a substantially similar manner as described with respect to the seventh embodiment. By the same token, in the polygonal alternative embodiment, the dedicated transmit elements and the dedicated receive elements are both placed in annular-like polygonal areas including a first annular-like area and a second annular-like area as described with respect to the seventh embodiment. Similarly, the third area, the fourth area and the fifth area also exist in the polygonal alternative embodiment in a substantially similar manner as described with respect to the seventh embodiment. Although the above alternative embodiments are not illustrated in drawings, the alternative embodiments are disclosed by the illustrated seventh embodiment in combination with the above description.

FIG. 8 is a diagram illustrating an eighth embodiment of the array in the probe according to the current invention. In general, the embodiment is a two-dimensional array 80 of transducer elements that includes transmit/receive elements that perform both transmit and receive functions, dedicated transmit elements that perform only transmit functions and functions and dedicated receive elements that perform only receive functions. That is, the embodiment according to the current invention includes shared transmit/receive elements that perform both transmit and receive functions within the same element in addition to the dedicated transmit elements and the dedicated receive elements. The dedicated transmit elements and the dedicated receive elements are interlaced with the transmit/receive elements in a certain predetermined spatial arrangement as indicated by different shades of color in the diagram.

The dedicated transmit elements and the dedicated receive elements are both placed in annular-like circular areas including a first annular-like area 81 and a second annular-like area 82 while the shared transmit/receive elements are placed in a sixth annular-like area 86. As indicated by different shades, the first annular-like area 81 exclusively includes either one of dedicated transmit elements or dedicated receive elements while the second annular-like area 82 area exclusively includes the other one of the dedicated transmit elements and the dedicated receive elements. In addition, the sixth annular-like area 86 include the shared transmit/receive elements. In other words, the first annular-like area 81 and the second annular-like area 82 alternate the dedicated transmit elements and the dedicated receive elements in their respective annular-like circular areas while the sixth annular-like area 86 is placed between the first annular-like area 81 and the second annular-like area 82 and includes the shared transmit/receive elements. For example, if the first annular-like area 81 exclusively includes the dedicated transmit elements, the second annular-like area 82 exclusively includes the dedicated receive elements and the sixth annular-like area 86 is placed between the first annular-like area 81 and the second annular-like area 82 and includes the shared transmit/receive elements. In the eighth embodiment, the second annular-like area 82 is immediately juxtaposed around the sixth annular-like area 86, and the sixth annular-like area 86 is immediately juxtaposed around the first annular-like area 81. Both the second annular-like area 82 and the sixth annular-like area 86 have a substantially concentric center with the first annular-like area 81.

As illustrated in the diagram, the first annular-like area 81 and the second annular-like area 82 are optionally repeated over a predetermined transducer surface of the two-dimensional array 80. As indicated by the shaded circular rings in the diagram, the additionally repeated annular-like areas 81A, 82A, 81B and 82B also exclusively have an alternate one of the dedicated transmit elements and the dedicated receive elements while the sixth annular-like areas 86, 86A and 86B include the shared transmit/receive elements. In the illustrated embodiment, as the second annular-like area 82 is larger than the first annular-like area 81 and is immediately juxtaposed around the sixth annular-like areas 86, the additionally repeated annular-like areas 81A, 82A, 81B and 82B and the sixth annular-like areas 86, 86A and 86B also have substantially the same spatial relationship among them. The term, “annular-like area” is intended to have the same meaning as already described with respect to FIG. 1 in the in the current patent application.

Still referring to FIG. 8, the exemplary embodiment additionally includes a third area 83 and a fourth area 84. The third area 83 is a circle and is located inside the first annular-like area 81 and at least over the concentric center. The third area 83 is optionally juxtaposed to the first annular-like area 81 or alternatively contained in the first annular-like area 81 with a gap between the third area 83 and the first annular-like area 81. In this embodiment, the third area 83 is indicated in white that the third area 83 is devoid of the dedicated transmit elements and the dedicated receive elements or is alternatively disabled. The third area 83 optionally further reduces the number of array elements and ultimately improves the cost, the power consumption and the size. The third area 83 also results in improved beam width and thereby enhances near-field lateral resolution in improving imaging quality. Since the third area 83 having non-functioning array elements or lacking array elements correlates with the opaque optical disk in a first Fresnel zone which produces the spot of Arago in optics diffraction theory, the third area 83 is also called Spot of Arago in the current application.

In contrast, the fourth area 84 is located outside the largest annular-like area 82B on the two-dimensional array surface. The fourth area 84 is optionally disabled or devoid of any functional transducer element. Alternatively, the fourth area 84 is optionally populated by the dedicated transmit elements for maximum power or the dedicated receive elements for maximum sensitivity.

In one exemplary array, the embodiment includes a total of ten thousand (10,000) array elements with 100 Azimuth elements and 100 Elevation elements. Among the 10,000 array elements, assuming that predetermined numbers M and N respectively indicate a number of dedicated transmit elements and dedicated receive elements while a third number O indicates a number of array elements that is unused, the sum of M+N+O is 10,000. For example, the first predetermined number M and the second predetermined number N are respectively 3750 dedicated transmit elements and 3750 dedicated receive elements while the third predetermined number O is 2600 unused array elements. Furthermore, based upon the above example, the 3750 dedicated transmit elements are optionally divided among the first annular-like areas 81, 81A and 81B whose area sizes are equal in one embodiment. In another embodiment, based upon the same example, the 3750 dedicated transmit elements are optionally divided among the first annular-like areas 81, 81A and 81B whose area sizes are not equal. By the same token, based upon the same example, the 3750 dedicated receive elements are optionally divided among the second annular-like areas 82, 82A and 82B whose area sizes may or may not be equal. In an alternative embodiment, the third area 83 is included in the number N if the third area 83 is equipped with array elements and unused.

In another exemplary embodiment, the array is optionally fully populated or sparsely populated by the dedicated transmit elements and the dedicated receive elements. In case of semi-sparsely populated rings, a predetermined Apodization function is applied to weight the detected signals for the purpose of shaping a beam profile.

In addition to the above illustrated embodiment, alternative embodiments based upon the eighth embodiment further include an elliptical embodiment and a polygonal embodiment. In the elliptical alternative embodiment, the shared transmit/receive elements, elements, the dedicated transmit elements and the dedicated receive elements are all placed in annular-like elliptical areas including a first annular-like area, a second annular-like area and a sixth annular-like area as described with respect to the eighth embodiment. Similarly, the third area and the fourth also exist in the elliptical alternative embodiment in a substantially similar manner as described with respect to the eighth embodiment. By the same token, in the polygonal alternative embodiment, the shared transmit/receive elements, the dedicated transmit elements and the dedicated receive elements are all placed in annular-like polygonal areas including a first annular-like area, a second annular-like area and a sixth annular-like area as described with respect to the eighth embodiment. Similarly, the third area and the fourth areas also exist in the polygonal alternative embodiment in a substantially similar manner as described with respect to the eighth embodiment. Although the above alternative embodiments are not illustrated in drawings, the alternative embodiments are disclosed by the illustrated eighth embodiment in combination with the above description.

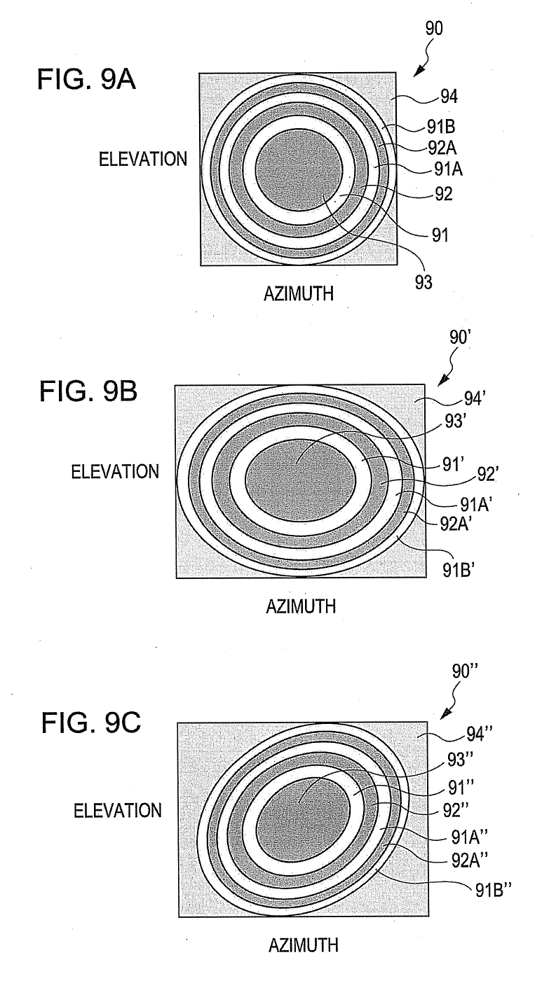

Now referring to FIGS. 9A, 9B and 9C, a certain optional operation of one of the above described embodiments will be described. FIG. 9A illustrates an embodiment of the array in the probe according to the current invention. In general, the embodiment is substantially the same as the first embodiment as illustrated in FIG. 1. A two-dimensional array 90 includes a first annular-like area 91 exclusively includes either one of dedicated transmit elements or dedicated receive elements while the second annular-like area 92 area exclusively includes the other one of the dedicated transmit elements and the dedicated receive elements. In other words, the first annular-like area 91 and the second annular-like area 92 alternate the dedicated transmit elements and the dedicated receive elements in their respective annular-like circular areas. For example, if the first annular-like area 91 exclusively includes the dedicated transmit elements, the second annular-like area 92 exclusively includes the dedicated receive elements. Furthermore, the second annular-like area 92 is immediately juxtaposed around the first annular-like area 91 and has a substantially concentric center with the first annular-like area 91.

As illustrated in the diagram, the first annular-like area 91 and the second annular-like area 92 are optionally repeated over a predetermined transducer surface of the two-dimensional array 90. As indicated by the shaded circular rings in the diagram, the additionally repeated annular-like areas 91A, 92A and 91B also exclusively have an alternate one of the dedicated transmit elements and the dedicated receive elements. In the illustrated embodiment, as the second annular-like area 92 is larger than the first annular-like area 91 and is immediately juxtaposed around the first annular-like area 91, the additionally repeated annular-like areas 91A, 92A and 91B also have substantially the same spatial relationship among them.

Still referring to FIG. 9A, the exemplary embodiment additionally includes a third area 93 and a fourth area 94. The third area 93 is located inside the first annular-like area 91 and at least over the concentric center. The third area 93 is optionally juxtaposed to the first annular-like area 91. In this embodiment, as the third area 93 is indicated by the same shade as the second annular-like area 92, the third area 93 exclusively includes the same one of the dedicated transmit elements and the dedicated receive elements as the second annular-like area 92. In contrast, the fourth area 94 is located outside the largest annular-like area 91B on the two-dimensional array surface. The fourth area 94 is optionally void of any functional transducer element or disabled. Alternatively, the fourth area 94 is optionally populated by the dedicated transmit elements for maximum power or the dedicated receive elements for maximum sensitivity.

The term, “annular-like area” is intended to mean the same as defined elsewhere in the current patent application. Since the definition is for the spatial relation of the array elements, it does not necessarily limit activation patterns or sequences of the dedicated transmit elements in transmitting the ultrasound pulses during the transmission operation. By the same token, the definition also does not necessarily limit activation patterns or sequences of the dedicated receive elements in detecting the ultrasound echoes during the receiving operation.

FIG. 9A also illustrates a certain activation pattern or sequence of the dedicated receive elements in detecting the ultrasound echoes during the receiving operation. During the receive operation, either one of the annular-like areas is activated to detect the ultrasound echoes. The activated annular-like area is optionally a combination of the first annular-like areas 91, 91A and 91B. Alternatively, a combination of the second annular-like area 92, 92A and the third area 93 is activated to detect the ultrasound echoes. In either case, the selected annular-like receive areas are dynamically activated or steered. In other words, the selected annular-like receive areas have the steering angle of 0 degrees. Thus, the annular-like receive areas substantially maintain their spatial relation of the dedicated receive elements.

Now referring to FIG. 9B, a diagram illustrates a certain activation pattern or sequence of the dedicated receive elements in detecting the ultrasound echoes during the receiving operation. During the receive operation, either one of the annular-like areas is activated to detect the ultrasound echoes. The activated annular-like area is optionally a combination of the first annular-like areas 91′, 91A′ and 91B′. Alternatively, a combination of the second annular-like area 92′, 92A′ and the third area 93′ is activated to detect the ultrasound echoes. In either case, the selected annular-like receive areas are dynamically activated or steered. In other words, the selected annular-like receive areas have the steering angle of 30 Azimuth degrees or 30 degrees in the X direction. Thus, the annular-like receive areas substantially elongated in their spatial relation of the dedicated receive elements. The annular-like receive areas become more elliptical in the direction of steering in comparison to the circular ring spatial relation of the dedicated receive elements.

Now referring to FIG. 9C, a diagram illustrates a certain activation pattern or sequence of the dedicated receive elements in detecting the ultrasound echoes during the receiving operation. During the receive operation, either one of the annular-like areas is activated to detect the ultrasound echoes. The activated annular-like area is optionally a combination of the first annular-like areas 91″, 91A″ and 91B″. Alternatively, a combination of the second annular-like area 92″, 92A″ and the third area 93″ is activated to detect the ultrasound echoes. In either case, the selected annular-like receive areas are dynamically activated or steered. In other words, the selected annular-like receive areas have the steering angle of 30 Azimuth degrees and 30 Elevation degrees or 30 degrees in the X and Y directions. Thus, the annular-like receive areas substantially elongated in their spatial relation of the dedicated receive elements. The annular-like receive areas become more elliptical in the direction of steering in comparison to the circular ring spatial relation of the dedicated receive elements.

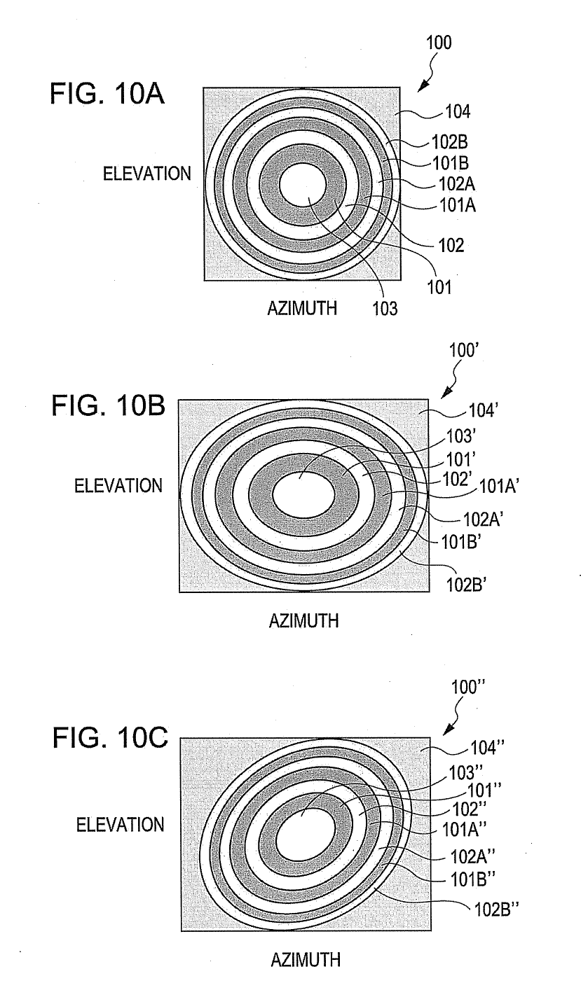

Now referring to FIGS. 10A, 10B and 10C, a certain optional operation of one of the above described embodiments will be described. FIG. 10A illustrates an embodiment of the array that is substantially the same as the fourth embodiment as illustrated in FIG. 4. A two-dimensional array 100 includes a first annular-like area 101 exclusively includes either one of dedicated transmit elements or dedicated receive elements while the second annular-like area 102 area exclusively includes the other one of the dedicated transmit elements and the dedicated receive elements. In other words, the first annular-like area 101 and the second annular-like area 102 alternate the dedicated transmit elements and the dedicated receive elements in their respective annular-like circular areas. For example, if the first annular-like area 101 exclusively includes the dedicated transmit elements, the second annular-like area 102 exclusively includes the dedicated receive elements. Furthermore, the second annular-like area 102 is immediately juxtaposed around the first annular-like area 101 and has a substantially concentric center with the first annular-like area 101.

As illustrated in the diagram, the first annular-like area 101 and the second annular-like area 102 are optionally repeated over a predetermined transducer surface of the two-dimensional array 100. As indicated by the shaded circular rings in the diagram, the additionally repeated annular-like areas 101A, 102A, 101B and 102B also exclusively have an alternate one of the dedicated transmit elements and the dedicated receive elements. In the illustrated embodiment, as the second annular-like area 102 is larger than the first annular-like area 101 and is immediately juxtaposed around the first annular-like area 101, area 101, the additionally repeated annular-like areas 101A, 102A, 101B and 102B also have substantially the same spatial relationship among them.

Still referring to FIG. 10A, the exemplary embodiment additionally includes a third area 103 and a fourth area 104. The third area 103 is a circle and is located inside the first annular-like area 101 and at least over the concentric center. The third area 103 is optionally juxtaposed to the first annular-like area 101 or alternatively contained in the first annular-like area 101 with a gap between the third area 103 and the first annular-like area 101. In this embodiment, the third area 103 is indicated in white that the third area 103 is devoid of the dedicated transmit elements and the dedicated receive elements or is alternatively disabled. The third area 103 optionally further reduces the number of array elements and ultimately improves the cost, the power consumption and the size. The third area 103 also results in improved beam width and thereby enhances near-field lateral resolution in improving imaging quality. Since the third area 103 having non-functioning array elements or lacking array elements correlates with the opaque optical disk in a first Fresnel zone which produces the spot of Arago in optics diffraction theory, the third area 103 is also called Spot of Arago in the current application.

In contrast, the fourth area 104 is located outside the largest annular-like area 102B on the two-dimensional array surface. The fourth area 104 is optionally void of any functional transducer element or disabled. Alternatively, the fourth area 104 is optionally populated by the dedicated transmit elements for maximum power or the dedicated receive elements for maximum sensitivity.

The term, “annular-like area” is intended to mean the same as defined elsewhere in the current patent application. Since the definition is for the spatial relation of the array elements, it does not necessarily limit activation patterns or sequences of the dedicated transmit elements in transmitting the ultrasound pulses during the transmission operation. By the same token, the definition also does not necessarily limit activation patterns or sequences of the dedicated receive elements in detecting the ultrasound echoes during the receiving operation.

FIG. 10A also illustrates a certain activation pattern or sequence of the dedicated receive elements in detecting the ultrasound echoes during the receiving operation. During the receive operation, either one of the annular-like areas is activated to detect the ultrasound echoes. The activated annular-like area is optionally a combination of the first annular-like areas 101, 101A and 101B. Alternatively, a combination of the second annular-like area 102, 102A and 102B is activated to detect the ultrasound echoes. In either case, the selected annular-like receive areas are dynamically activated or steered. In other words, the selected annular-like receive areas have the steering angle of 0 degrees. Thus, the annular-like receive areas substantially maintain their spatial relation of the dedicated receive elements.

Now referring to FIG. 10B, a diagram illustrates a certain activation pattern or sequence of the dedicated receive elements of the same embodiment as described with respect to FIG. 10A in detecting the ultrasound echoes during the receiving operation. During the receive operation, either one of the annular-like areas is activated to detect the ultrasound echoes. The activated annular-like area is optionally a combination of the first annular-like areas 101′, 101A′ and 101B′. Alternatively, a combination of the second annular-like area 102′, 102A′ and 102B′ is activated to detect the ultrasound echoes. In either case, the selected annular-like receive areas are dynamically activated or steered. In other words, the selected annular-like receive areas have the steering angle of 30 Azimuth degrees or 30 degrees in the X direction. Thus, the annular-like receive areas substantially elongated in their spatial relation of the dedicated receive elements. The annular-like receive areas become more elliptical in the direction of steering in comparison to the circular ring spatial relation of the dedicated receive elements.

Now referring to FIG. 10C, a diagram illustrates a certain activation pattern or sequence of the dedicated receive elements of the same embodiment as described with respect to FIG. 10A in detecting the ultrasound echoes during the receiving operation. During the receive operation, either one of the annular-like areas is activated to detect the ultrasound echoes. The activated annular-like area is optionally a combination of the first annular-like areas 101″, 101A″ and 101B″. Alternatively, a combination of the second annular-like area 102″, 102A″ and 102B″ is activated to detect the ultrasound echoes. In either case, the selected annular-like receive areas are dynamically activated or steered. In other words, the selected annular-like receive areas have the steering angle of 30 Azimuth degrees and 30 Elevation degrees or 30 degrees in the X and Y directions. Thus, the annular-like receive areas substantially elongated in their spatial relation of the dedicated receive elements. The annular-like receive areas become more elliptical in the direction of steering in comparison to the circular ring spatial relation of the dedicated receive elements.

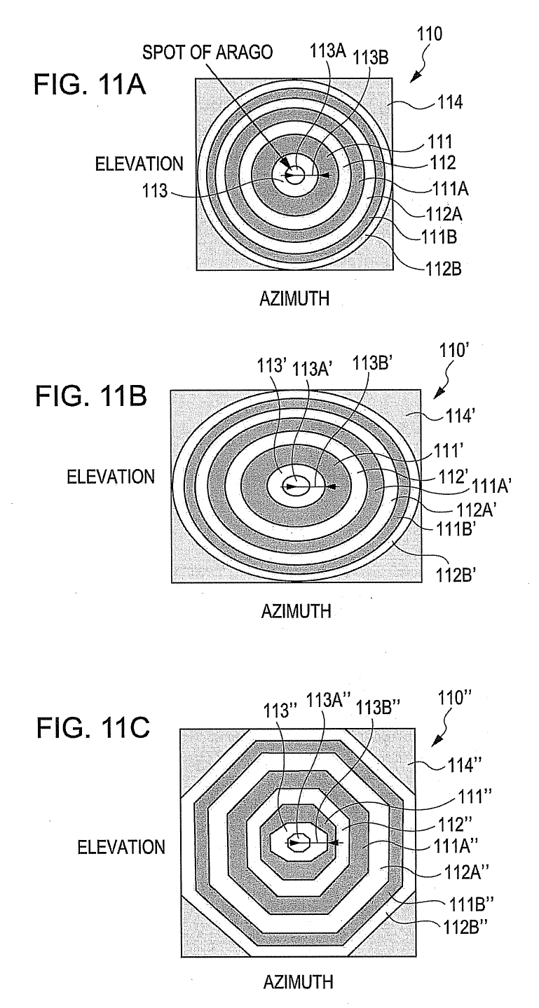

Now referring to FIG. 11A, a diagram illustrates an embodiment that is substantially the same as the fourth embodiment as illustrated in FIG. 4. A two-dimensional array 110 includes a first annular-like area 111 exclusively includes either one of dedicated transmit elements or dedicated receive elements while the second annular-like area 112 area exclusively includes the other one of the dedicated transmit elements and the dedicated receive elements. In other words, the first annular-like area 111 and the second annular-like area 112 alternate the dedicated transmit elements and the dedicated receive elements in their respective annular-like circular areas. For example, if the first annular-like area 111 exclusively includes the dedicated transmit elements, the second annular-like area 112 exclusively includes the dedicated receive elements. Furthermore, the second annular-like area 112 is immediately juxtaposed around the first annular-like area 111 and has a substantially concentric center with the first annular-like area 111.

As illustrated in the diagram, the first annular-like area 111 and the second annular-like area 112 are optionally repeated over a predetermined transducer surface of the two-dimensional array 110. As indicated by the shaded circular rings in the diagram, the additionally repeated annular-like areas 111A, 112A, 111B and 112B also exclusively have an alternate one of the dedicated transmit elements and the dedicated receive elements. In the illustrated embodiment, as the second annular-like area 112 is larger than the first the first annular-like area 111 and is immediately juxtaposed around the first annular-like area 111, the additionally repeated annular-like areas 111A, 112A, 111B and 112B also have substantially the same spatial relationship among them.

Still referring to FIG. 11A, the exemplary embodiment additionally includes a third area 113 and a fourth area 114. The third area 113 is a circle and is located inside the first annular-like area 111 and at least over the concentric center. The third area 113 is optionally juxtaposed to the first annular-like area 111 or alternatively contained in the first annular-like area 111 with a gap between the third area 113 and the first annular-like area 111. In this embodiment, the third area 113 is indicated in white that the third area 113 is devoid of the dedicated transmit elements and the dedicated receive elements or is alternatively disabled. The third area 113 optionally further reduces the number of array elements and ultimately improves the cost, the power consumption and the size. The third area 113 also results in improved beam width and thereby enhances near-field lateral resolution in improving imaging quality. Since the third area 113 having non-functioning array elements or lacking array elements correlates with the opaque optical disk in a first Fresnel zone which produces the spot of Arago in optics diffraction theory, the third area 113 is also called Spot of Arago in the current application.

In contrast, the fourth area 114 is located outside the largest annular-like area 112B on the two-dimensional array surface. The fourth area 114 is optionally void of any functional transducer element or disabled. Alternatively, the fourth area 114 is optionally populated by the dedicated transmit elements for maximum power or the dedicated receive elements for maximum sensitivity.

The term, “annular-like area” is intended to mean the same as defined elsewhere in the current patent application. Since the definition is for the spatial relation of the array elements, it does not necessarily limit activation patterns or sequences of the dedicated transmit elements in transmitting the ultrasound pulses during the transmission operation. By the same token, the definition also does not necessarily limit activation patterns or sequences of the dedicated receive elements in detecting the ultrasound echoes during the receiving operation.

FIG. 11A also illustrates a certain activation pattern or sequence of the dedicated receive elements in detecting the ultrasound echoes during the receiving operation. During the receive operation, either one of the annular-like areas is activated to detect the ultrasound echoes. The activated annular-like area is optionally a combination of the first annular-like areas 111, 111A and 111B. Alternatively, a combination of the second annular-like area 112, 112A and 112E is activated to detect the ultrasound echoes. In either case, the selected annular-like receive areas are neither dynamically activated nor steered. Thus, the annular-like receive areas substantially maintain their spatial relation of the dedicated receive elements.

In another exemplary embodiment, the array is optionally fully populated or sparsely populated by the dedicated transmit elements and the dedicated receive elements. In case of semi-sparsely populated rings, a predetermined apodization function is applied to weight the detected signals for the purpose of shaping a beam profile.

On the other hand, the third area or Spot of Arago 113 is dynamic during the receive operation. For example, the size of Spot of Arago 113 dynamically changes from a first size 113A to a second size 113B or vice versa during the receive operation. The size change is not limited to the above two sizes and includes an exemplary size sequence of small to large to smaller to none. For example, the size of the dynamic Spot of Arago 113 changes due to the first annular-like areas 111, which changes its size by activating or deactivating predetermined portions in a certain sequence during the receive operation. Furthermore, the Spot of Arago 113 dynamically changes with respect to image depth or time in a certain embodiment.

Now referring to FIG. 11B, a diagram illustrates an embodiment that is substantially the same as the fifth embodiment as illustrated in FIG. 5. A two-dimensional array 110′ includes a first annular-like area 111′ exclusively includes either one of dedicated transmit elements or dedicated receive elements while the second annular-like area 112′ area exclusively includes the other one of the dedicated transmit elements and the dedicated receive elements. In other words, the first annular-like area 111′ and the second annular-like area 112′ alternate the dedicated transmit elements and the dedicated receive elements in their respective annular-like elliptical areas. For example, if the first annular-like area 111′ exclusively includes the dedicated transmit elements, the second annular-like area 112′ exclusively includes the dedicated receive elements. Furthermore, the second annular-like area 112′ is immediately juxtaposed around the first annular-like area 111′ and has a substantially concentric center with the first annular-like area 111′.

As illustrated in the diagram, the first annular-like area 111′ and the second annular-like area 112′ are optionally repeated over a predetermined transducer surface of the two-dimensional array 110′. As indicated by the shaded elliptical rings in the diagram, the additionally repeated annular-like areas 111A′, 112A′, 111B′ and 112B′ also exclusively have an alternate one of the dedicated transmit elements and the dedicated receive elements. In the illustrated embodiment, as the second annular-like area 112′ is larger than the first annular-like area 111′ and is immediately juxtaposed around the first annular-like area 111′, the additionally repeated annular-like areas 111A′, 112A′, 111B′ and 112B′ also have substantially the same spatial relationship among them.

Still referring to FIG. 11B, the exemplary embodiment additionally includes a third area 113′ and a fourth area 114′. The third area 113′ is a ellipse and is located inside the first annular-like area 111′ and at least over the concentric center. The third area 113′ is optionally juxtaposed to the first annular-like area 111′ or alternatively contained in the first annular-like area 111′ with a gap between the third area 113′ and the first annular-like area 111′. In this embodiment, the third area 113′ is indicated in white that the third area 113′ is devoid of the dedicated transmit elements and the dedicated receive elements or is alternatively disabled. The third area 113′ optionally further reduces the number of array elements and ultimately improves the cost, the power consumption and the size. The third area 113′ also results in improved beam width and thereby enhances near-field lateral resolution in improving imaging quality. Since the third area 113′ having non-functioning array elements or lacking array elements correlates with the opaque optical disk in a first Fresnel zone which produces the spot of Arago in optics diffraction theory, the third area 113′ is also called Spot of Arago in the current application.

In contrast, the fourth area 114′ is located outside the largest annular-like area 112B′ on the two-dimensional array surface. The fourth area 114′ is optionally void of any functional transducer element or disabled. Alternatively, the fourth area 114′ is optionally populated by the dedicated transmit elements for maximum power or the dedicated receive elements for maximum sensitivity.

The term, “annular-like area” is intended to mean the same as defined elsewhere in the current patent application. Since the definition is for the spatial relation of the array elements, it does not necessarily limit activation patterns or sequences of the dedicated transmit elements in transmitting the ultrasound pulses during the transmission operation. By the same token, the definition also does not necessarily limit activation patterns or sequences of the dedicated receive elements in detecting the ultrasound echoes during the receiving operation.

FIG. 11B also illustrates a certain activation pattern or sequence of the dedicated receive elements in detecting the ultrasound echoes during the receiving operation. During the receive operation, either one of the annular-like areas is activated to detect the ultrasound echoes. The activated annular-like area is optionally a combination of the first annular-like areas 111′, 111A′ and 111B′. Alternatively, a combination of the second annular-like area 112′, 112A′ and 112B′ is activated to detect the ultrasound echoes. In either case, the selected annular-like receive areas are neither dynamically activated nor steered. Thus, the annular-like receive areas substantially maintain their spatial relation of the dedicated receive elements.

In another exemplary embodiment, the array is optionally fully populated or sparsely populated by the dedicated transmit elements and the dedicated receive elements. In case of semi-sparsely populated rings, a predetermined Apodization function is applied to weight the detected signals for the purpose of shaping a beam profile.

On the other hand, the third area or Spot of Arago 113′ is dynamic during the receive operation. For example, the size of Spot of Arago 113′ dynamically changes from a first size 113A′ to a second size 113B′ or vice versa during the receive operation. The size change is not limited to the above two sizes and includes an exemplary size sequence of small to large to smaller to none. For example, the size of the dynamic Spot of Arago 113′ changes due to the first annular-like areas 111′, which changes its size by activating or deactivating predetermined portions in a certain sequence during the receive operation. Furthermore, the Spot of Arago 113′ dynamically changes with respect to image depth or time in a certain embodiment.

Now referring to FIG. 11C, a diagram illustrates an embodiment that is substantially the same as the sixth embodiment as illustrated in FIG. 6. A two-dimensional array 110″ includes a first annular-like area 111″ exclusively includes either one of dedicated transmit elements or dedicated receive elements while the second annular-like area 112″ area exclusively includes the other one of the dedicated transmit elements and the dedicated receive elements. In other words, the first annular-like area 111″ and the second annular-like area 112″ alternate the dedicated transmit elements and the dedicated receive elements in their respective annular-like polygonal areas. For example, if the first annular-like area 111″ exclusively includes the dedicated transmit elements, the second annular-like area 112″ exclusively includes the dedicated receive elements. Furthermore, the second annular-like area 112″ is immediately juxtaposed around the first annular-like area 111″ and has a substantially concentric center with the first annular-like area 111″.

As illustrated in the diagram, the first annular-like area 111″ and the second annular-like area 112″ are optionally repeated over a predetermined transducer surface of the the two-dimensional array 110″. As indicated by the shaded polygonal rings in the diagram, the additionally repeated annular-like areas 111A″, 112A″, 111B″ and 112B″ also exclusively have an alternate one of the dedicated transmit elements and the dedicated receive elements. In the illustrated embodiment, as the second annular-like area 112″ is larger than the first annular-like area 111″ and is immediately juxtaposed around the first annular-like area 111″, the additionally repeated annular-like areas 111A″, 112A″, 111B″ and 112B″ also have substantially the same spatial relationship among them.

Still referring to FIG. 11C, the exemplary embodiment additionally includes a third area 113″ and a fourth area 114″. The third area 113″ is a polygon and is located inside the first annular-like area 111″ and at least over the concentric center. The third area 113″ is optionally juxtaposed to the first annular-like area 111″ or alternatively contained in the first annular-like area 111″ with a gap between the third area 113″ and the first annular-like area 111″. In this embodiment, the third area 113″ is indicated in white that the third area 113′ is devoid of the dedicated transmit elements and the dedicated receive elements or is alternatively disabled. The third area 113″ optionally further reduces the number of array elements and ultimately improves the cost, the power consumption and the size. The third area 113″ also results in improved beam width and thereby enhances near-field lateral resolution in improving imaging quality. Since the third area 113″ having non-functioning array elements or lacking array elements correlates with the opaque optical disk in a first Fresnel zone which produces the spot of Arago in optics diffraction theory, the third area 113″ is also called Spot of Arago in the current application.

In contrast, the fourth area 114″ is located outside the largest annular-like area 112B″ on the two-dimensional array surface. The fourth area 114″ is optionally void of any functional transducer element or disabled. Alternatively, the fourth area 114″ is optionally populated by the dedicated transmit elements for maximum power or the dedicated receive elements for maximum sensitivity.

The term, “annular-like area” is intended to mean the same as defined elsewhere in in the current patent application. Since the definition is for the spatial relation of the array elements, it does not necessarily limit activation patterns or sequences of the dedicated transmit elements in transmitting the ultrasound pulses during the transmission operation. By the same token, the definition also does not necessarily limit activation patterns or sequences of the dedicated receive elements in detecting the ultrasound echoes during the receiving operation.

FIG. 11C also illustrates a certain activation pattern or sequence of the dedicated receive elements in detecting the ultrasound echoes during the receiving operation. During the receive operation, either one of the annular-like areas is activated to detect the ultrasound echoes. The activated annular-like area is optionally a combination of the first annular-like areas 111″, 111A″ and 111B″. Alternatively, a combination of the second annular-like area 112″, 112A″ and 112B″ is activated to detect the ultrasound echoes. In either case, the selected annular-like receive areas are neither dynamically activated nor steered. Thus, the annular-like receive areas substantially maintain their spatial relation of the dedicated receive elements.

In another exemplary embodiment, the array is optionally fully populated or sparsely populated by the dedicated transmit elements and the dedicated receive elements. In case of semi-sparsely populated rings, a predetermined Apodization function is applied to weight the detected signals for the purpose of shaping a beam profile.

On the other hand, the third area or Spot of Arago 113″ is dynamic during the receive operation. For example, the size of Spot of Arago 113″ dynamically changes from a first size 113A″ to a second size 113B″ or vice versa during the receive operation. The size change is not limited to the above two sizes and includes an exemplary size sequence of small to large to smaller to none. For example, the size of the dynamic Spot of Arago 113″ changes due to the first annular-like areas 111″, which changes its size by activating or deactivating predetermined portions in a certain sequence during the receive operation. Furthermore, the Spot of Arago 113″ dynamically changes with respect to image depth or time in a certain embodiment.

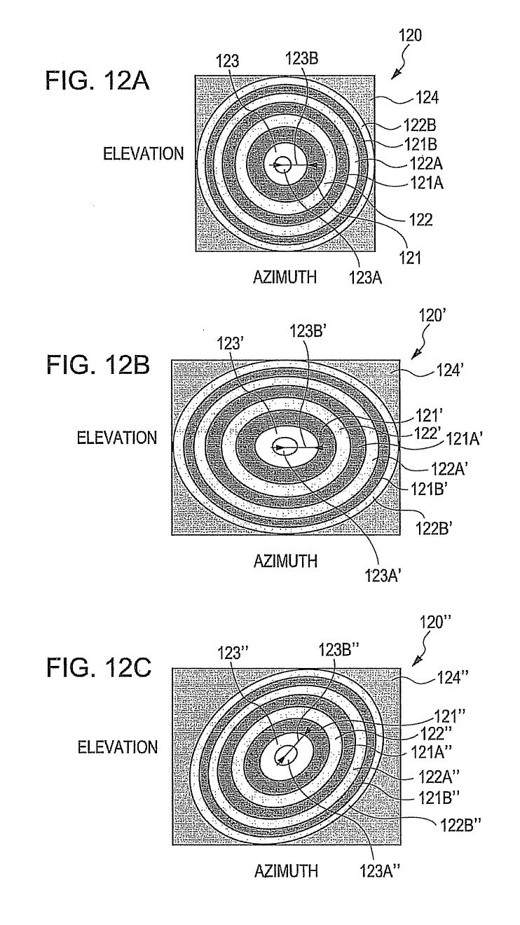

Now referring to FIGS. 12A, 12B and 12C, a certain optional operation of one of the above described embodiments will be described. FIG. 12A illustrates an embodiment of the array that is substantially the same as a combination of the embodiments as illustrated in FIGS. 10A and 11A. A two-dimensional array 120 includes a first annular-like area 121 exclusively includes either one of dedicated transmit elements or dedicated receive elements while the second annular-like area 122 area exclusively includes the other one of the dedicated transmit elements and the dedicated receive elements. In other words, the first annular-like area 121 and the second annular-like area 122 alternate the dedicated transmit elements and the dedicated receive elements in their respective annular-like circular areas. For example, if the first annular-like area 121 exclusively includes the dedicated transmit elements, the second annular-like area 122 exclusively includes the dedicated receive elements. Furthermore, the second annular-like area 122 is immediately juxtaposed around the first annular-like area 121 and has a substantially concentric center with the first annular-like area 121.

As illustrated in the diagram, the first annular-like area 121 and the second annular-like area 122 are optionally repeated over a predetermined transducer surface of the two-dimensional array 120. As indicated by the shaded circular rings in the diagram, the additionally repeated annular-like areas 121A, 122A, 121B and 122B also exclusively have an alternate one of the dedicated transmit elements and the dedicated receive elements. In the illustrated embodiment, as the second annular-like area 122 is larger than the first annular-like area 121 and is immediately juxtaposed around the first annular-like area 121, the additionally repeated annular-like areas 121A, 122A, 121B and 122B also have substantially the same spatial relationship among them.

Still referring to FIG. 12A, the exemplary embodiment additionally includes a third area 123 and a fourth area 124. The third area 123 is a circle and is located inside the the first annular-like area 121 and at least over the concentric center. The third area 123 is optionally juxtaposed to the first annular-like area 121 or alternatively contained in the first annular-like area 121 with a gap between the third area 123 and the first annular-like area 121. In this embodiment, the third area 123 is indicated in white that the third area 123 is devoid of the dedicated transmit elements and the dedicated receive elements or is alternatively disabled. The third area 123 optionally further reduces the number of array elements and ultimately improves the cost, the power consumption and the size. The third area 123 also results in improved beam width and thereby enhances near-field lateral resolution in improving imaging quality. Since the third area 123 having non-functioning array elements or lacking array elements correlates with the opaque optical disk in a first Fresnel zone which produces the spot of Arago in optics diffraction theory, the third area 123 is also called Spot of Arago in the current application.

In contrast, the fourth area 124 is located outside the largest annular-like area 122B on the two-dimensional array surface. The fourth area 124 is optionally void of any functional transducer element or disabled. Alternatively, the fourth area 124 is optionally populated by the dedicated transmit elements for maximum power or the dedicated receive elements for maximum sensitivity.

The term, “annular-like area” is intended to mean the same as defined elsewhere in the current patent application. Since the definition is for the spatial relation of the array elements, it does not necessarily limit activation patterns or sequences of the dedicated transmit elements in transmitting the ultrasound pulses during the transmission operation. By the same token, the definition also does not necessarily limit activation patterns or sequences of the dedicated receive elements in detecting the ultrasound echoes during the receiving operation.

FIG. 12A also illustrates a certain activation pattern or sequence of the dedicated receive elements in detecting the ultrasound echoes during the receiving operation. During the receive operation, either one of the annular-like areas is activated to detect the ultrasound echoes. The activated annular-like area is optionally a combination of the first annular-like areas 121, 121A and 121B. Alternatively, a combination of the second annular-like area 122, 122A and 122B is activated to detect the ultrasound echoes. In either case, the selected annular-like receive areas are dynamically activated or steered. In other words, the selected annular-like receive areas have the steering angle of 0 degrees. Thus, the annular-like receive areas substantially maintain their spatial relation of the dedicated receive elements.

At the same time, the third area or Spot of Arago 123 is dynamic during the receive operation. For example, the size of Spot of Arago 123 dynamically changes from a first size 123A to a second size 123B or vice versa during the receive operation. For example, the size of the dynamic Spot of Arago 123 changes due to the first annular-like areas 121, which changes its size by activating or deactivating predetermined portions in a certain sequence during the receive operation. Furthermore, the Spot of Arago 123 dynamically changes with respect to image depth or time in a certain embodiment.

Now referring to FIG. 12B, a diagram illustrates a certain activation pattern or sequence of the dedicated receive elements of the same embodiment as described with respect to FIG. 12A in detecting the ultrasound echoes during the receiving operation. During the receive operation, either one of the annular-like areas is activated to detect the ultrasound echoes. The activated annular-like area is optionally a combination of the first annular-like areas 121′, 121A′ and 121B′. Alternatively, a combination of the second annular-like area 122′, 122A′ and 122B′ is activated to detect the ultrasound echoes. In either case, the selected annular-like receive areas are dynamically activated or steered. In other words, the selected annular-like receive areas have the steering angle of 30 Azimuth degrees or 30 degrees in the X direction. Thus, the annular-like receive areas substantially elongated in their spatial relation of the dedicated receive elements. The annular-like receive areas become more elliptical in the direction of steering in comparison to the circular ring spatial relation of the dedicated receive elements.

At the same time, the third area or Spot of Arago 123′ is dynamic during the receive operation. For example, the size of Spot of Arago 123′ dynamically changes from a first size 123A′ to a second size 123B′ or vice versa during the receive operation. For example, the size of the dynamic Spot of Arago 123′ changes due to the first annular-like areas 121′, which changes its size by activating or deactivating predetermined portions in a certain sequence during the receive operation. Furthermore, the Spot of Arago 123′ dynamically changes with respect to image depth or time in a certain embodiment.

Now referring to FIG. 12C, a diagram illustrates a certain activation pattern or sequence of the dedicated receive elements of the same embodiment as described with respect to FIG. 12A in detecting the ultrasound echoes during the receiving operation. During the receive operation, either one of the annular-like areas is activated to detect the ultrasound echoes. The activated annular-like area is optionally a combination of the first annular-like areas 121″, 121A″ and 121B″. Alternatively, a combination of the second annular-like area 122″, 122A″ and 122B″ is activated to detect the ultrasound echoes. In either case, the selected annular-like receive areas are dynamically activated or steered. In other words, the selected annular-like receive areas have the steering angle of 30 Azimuth degrees and 30 Elevation degrees or 30 degrees in the X and Y directions. Thus, the annular-like receive areas substantially elongated in their spatial relation of the dedicated receive elements. The annular-like receive areas become more elliptical in the direction of steering in comparison to the circular ring spatial relation of the dedicated receive elements.

At the same time, the third area or Spot of Arago 123″ is dynamic during the receive operation. For example, the size of Spot of Arago 123″ dynamically changes from a first size 123A″ to a second size 123B″ or vice versa during the receive operation. For example, the size of the dynamic Spot of Arago 123″ changes due to the first annular-like areas 121″, which changes its size by activating or deactivating predetermined portions in a certain sequence during the receive operation. Furthermore, the Spot of Arago 123″ dynamically changes with respect to image depth or time in a certain embodiment.

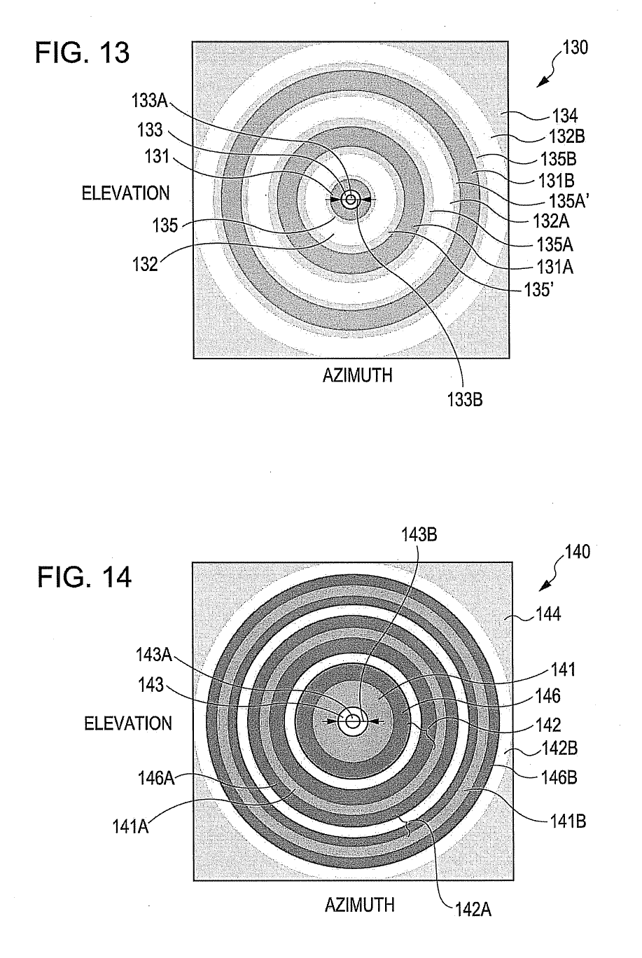

FIG. 13 is a diagram illustrating an embodiment of the array in the probe according to the current invention. In general, the embodiment is substantially the same as the seventh embodiment as illustrated in FIG. 7. In general, the embodiment is a two-dimensional array 130 of transducer elements that includes dedicated transmit elements that perform only transmit functions and dedicated receive elements that perform only receive functions. That is, the embodiment according to the current invention excludes any shared transmit/receive elements that perform both transmit and receive functions within the same element. The dedicated transmit elements and the dedicated receive elements are placed in a certain predetermined spatial arrangement as indicated by different shades of color in the diagram.

The dedicated transmit elements and the dedicated receive elements are both placed in annular-like circular areas including a first annular-like area 131 and a second annular-like area 132. As indicated by different shades, the first annular-like area 131 exclusively includes either one of dedicated transmit elements or dedicated receive elements while the second annular-like area 132 area exclusively includes the other one of the dedicated transmit elements and the dedicated receive elements. In other words, the first annular-like area 131 and the second annular-like area 132 alternate the dedicated transmit elements and the dedicated receive elements in their respective annular-like circular areas. For example, if the first annular-like area 131 exclusively includes the dedicated transmit elements, the second annular-like area 132 exclusively includes the dedicated receive elements. Although the second annular-like area 132 is not immediately juxtaposed around the first annular-like area 131, the second annular-like area 132 has a substantially concentric center with the first annular-like area 131.

In the embodiment of the array in the probe, there is an optional annular-like area 135 between the first annular-like area 131 and the second annular-like area 132. The optional annular-like area 135 is optionally populated with either one of the dedicated transmit elements or the dedicated receive elements, and these elements may be also optionally used or disabled. Alternatively, the optional annular-like area 135 is optionally populated with neither one of the dedicated transmit elements or the dedicated receive elements. Furthermore, an additional optional annular-like area 135′ surrounds the second annular-like area 132, and the additional optional annular-like area 135′ may be implemented in a similar manner as the optional annular-like area 135.

As illustrated in the diagram, the first annular-like area 131 and the second annular-like area 132 are optionally repeated over a predetermined transducer surface of the two-dimensional array 130. As indicated by the shaded circular rings in the diagram, the additionally repeated annular-like areas 131A, 132A, 131B and 132B also exclusively have an alternate one of the dedicated transmit elements and the dedicated receive elements. In the illustrated embodiment, as the second annular-like area 132 is larger than the first annular-like area 131 and is not immediately juxtaposed around the first annular-like area 131, the additionally repeated annular-like areas 131A, 132A, 131B and 132B also have substantially the same spatial relationship among them. By the same token, the additionally repeated annular-like areas 131A, 132A, 131B and 132B are interlaced by optional annular-like areas 135A and 135B as well as by additional optional annular-like area 135A′. The term, “annular-like area” is intended to have the same meaning as already described with respect to FIG. 1 in the in the current patent application.

Still referring to FIG. 13, the exemplary embodiment additionally includes a third area 133 and a fourth area 134. The third area 133 is a circle and is located inside the first annular-like area 131 and at least over the concentric center. The third area 133 is optionally juxtaposed to the first annular-like area 131 or alternatively contained in the first annular-like area 131 with a gap between the third area 133 and the first annular-like area 131. In this embodiment, the third area 133 is indicated in white that the third area 133 is devoid of the dedicated transmit elements and the dedicated receive elements or is alternatively disabled. The third area 133 optionally further reduces the number of array elements and ultimately improves the cost, the power consumption and the size. The third area 133 also results in improved beam width and thereby enhances near-field lateral resolution in improving imaging quality. Since the third area 133 having non-functioning array elements or lacking array elements correlates with the opaque optical disk in a first Fresnel zone which produces the spot of Arago in optics diffraction theory, the third area 133 is also called Spot of Arago in the current application.

In contrast, the fourth area 134 is located outside the largest annular-like area 132B on the two-dimensional array surface. The fourth area 134 is optionally disabled or devoid of any functional transducer element. Alternatively, the fourth area 134 is optionally populated by the dedicated transmit elements for maximum power or the dedicated receive elements for maximum sensitivity.

In another exemplary embodiment, the array is optionally fully populated or sparsely populated by the dedicated transmit elements and the dedicated receive elements. In case of semi-sparsely populated rings, a predetermined Apodization function is applied to weight the detected signals for the purpose of shaping a beam profile.

FIG. 13 also illustrates a certain activation pattern or sequence of the dedicated receive elements in detecting the ultrasound echoes during the receiving operation. During the receive operation, either one of the annular-like areas is activated to detect the ultrasound echoes. The activated annular-like area is optionally a combination of the first annular-like areas 131, 131A and 131B. Alternatively, a combination of the second annular-like area 132, 132A and 132B is activated to detect the ultrasound echoes. In either case, the selected annular-like receive areas are neither dynamically activated nor steered. Thus, the annular-like receive areas substantially maintain their spatial relation of the dedicated receive elements.

On the other hand, the third area or Spot of Arago 133 is dynamic during the receive operation. For example, the size of Spot of Arago 133 dynamically changes from a first size 133A to a second size 133B or vice versa during the receive operation. For example, the size of the dynamic Spot of Arago 133 changes due to the first annular-like areas 131, which changes its size by activating or deactivating predetermined portions in a certain sequence during the receive operation. Furthermore, the Spot of Arago 133 dynamically changes with respect to image depth or time in a certain embodiment.

In addition to the above illustrated embodiment, alternative embodiments based upon the above embodiment further include an elliptical embodiment and a polygonal embodiment. In the elliptical alternative embodiment, the dedicated transmit elements and the dedicated receive elements are both placed in annular-like elliptical areas including a first annular-like area and a second annular-like area as described with respect to the seventh embodiment. Similarly, the third area, the fourth and the fifth area also exist in the elliptical alternative embodiment in a substantially similar manner as described with respect to the above embodiment. By the same token, in the polygonal alternative embodiment, the dedicated transmit elements and the dedicated receive elements are both placed in annular-like polygonal areas including a first annular-like area and a second annular-like area as described with respect to the above embodiment. Similarly, the third area, the fourth area and the fifth area also exist in the polygonal alternative embodiment in a substantially similar manner as described with respect to the above illustrated embodiment. Although the above alternative embodiments are not illustrated in drawings, the alternative embodiments are disclosed by the illustrated embodiment in combination with the above description. The operation of these alternative embodiments is also substantially similar to the above described embodiment.

FIG. 14 is a diagram illustrating a ninth embodiment of the array in the probe according to the current invention. In general, the embodiment is substantially the same as the eighth embodiment as illustrated in FIG. 8. In general, the embodiment is a two-dimensional array 140 of transducer elements that includes transmit/receive elements that perform both transmit and receive functions, dedicated transmit elements that perform only transmit functions and dedicated receive elements that perform only receive functions. That is, the embodiment according to the current invention includes shared transmit/receive elements that perform both transmit and receive functions within the same element in addition to the dedicated transmit elements and the dedicated receive elements. The [symbol] The dedicated transmit elements and the dedicated receive elements are interlaced with the transmit/receive elements in a certain predetermined spatial arrangement as indicated by different shades of color in the diagram.

The dedicated transmit elements and the dedicated receive elements are both placed in annular-like circular areas including a first annular-like area 141 and a second annular-like area 142 while the shared transmit/receive elements are placed in a sixth annular-like area 146. As indicated by different shades, the first annular-like area 141 exclusively includes either one of dedicated transmit elements or dedicated receive elements while the second annular-like area 142 area exclusively includes the other one of the dedicated transmit elements and the dedicated receive elements. In addition, the sixth annular-like area 146 include the shared transmit/receive elements. In other words, the first annular-like area 141 and the second annular-like area 142 alternate the dedicated transmit elements and the dedicated receive elements in their respective annular-like circular areas while the sixth annular-like area 146 is placed between the first annular-like area 141 and the second annular-like area 142 and includes the shared transmit/receive elements. For example, if the first annular-like area 141 exclusively includes the dedicated transmit elements, the second annular-like area 142 exclusively includes the dedicated receive elements and the sixth annular-like area 146 is placed between the first annular-like area 141 and the second annular-like area 142 and includes the shared transmit/receive elements. In the eighth embodiment, the second annular-like area 142 is immediately juxtaposed around the sixth annular-like area 146, and the sixth annular-like area 146 is immediately juxtaposed around the first annular-like area 141. Both the second annular-like area 142 and the sixth annular-like area 146 have a substantially concentric center with the first annular-like area 141.

As illustrated in the diagram, the first annular-like area 141 and the second annular-like area 142 are optionally repeated over a predetermined transducer surface of the two-dimensional array 140. As indicated by the shaded circular rings in the diagram, the additionally repeated annular-like areas 141A, 142A, 141B and 142B also exclusively have have an alternate one of the dedicated transmit elements and the dedicated receive elements while the sixth annular-like areas 146, 146A and 146B include the shared transmit/receive elements. In the illustrated embodiment, as the second annular-like area 142 is larger than the first annular-like area 141 and is immediately juxtaposed around the sixth annular-like areas 146, the additionally repeated annular-like areas 141A, 142A, 141B and 142B and the sixth annular-like areas 146, 146A and 146B also have substantially the same spatial relationship among them. The term, “annular-like area” is intended to have the same meaning as already described with respect to FIG. 1 in the in the current patent application.

Still referring to FIG. 14, the exemplary embodiment additionally includes a third area 143 and a fourth area 144. The third area 143 is a circle and is located inside the first annular-like area 141 and at least over the concentric center. The third area 143 is optionally juxtaposed to the first annular-like area 141 or alternatively contained in the first annular-like area 141 with a gap between the third area 143 and the first annular-like area 141. In this embodiment, the third area 143 is indicated in white that the third area 143 is devoid of the dedicated transmit elements and the dedicated receive elements or is alternatively disabled. The third area 143 optionally further reduces the number of array elements and ultimately improves the cost, the power consumption and the size. The third area 143 also results in improved beam width and thereby enhances near-field lateral resolution in improved imaging quality. Since the third area 143 having non-functioning array elements or lacking array elements correlates with the opaque optical disk in a first Fresnel zone which produces the spot of Arago in optics diffraction theory, the third area 143 is also called Spot of Arago in the current application.

In contrast, the fourth area 144 is located outside the largest annular-like area 142B on the two-dimensional array surface. The fourth area 144 is optionally disabled or devoid of any functional transducer element. Alternatively, the fourth area 144 is optionally populated by the dedicated transmit elements for maximum power or the dedicated receive elements for maximum sensitivity.

In one exemplary array, the embodiment includes a total of ten thousand (10,000) array elements with 100 Azimuth elements and 100 Elevation elements. Among the 10,000 array elements, assuming that predetermined numbers M and N respectively indicate a number of dedicated transmit elements and dedicated receive elements while a third number O indicates a number of array elements that is unused, the sum of M+N+O is 10,000. For example, the first predetermined number M and the second predetermined number N are respectively 3750 dedicated transmit elements and 3750 dedicated receive elements while the third predetermined number O is 2600 unused array elements. Furthermore, based upon the above example, the 3750 dedicated transmit elements are optionally divided among the first annular-like areas 141, 141A and 141B whose area sizes are equal in one embodiment. In another embodiment, based upon the same example, the 3750 dedicated transmit elements are optionally divided among the first annular-like areas 141, 141A and 141B whose area sizes are not equal. By the same token, based upon the same example, the 3750 dedicated receive elements are optionally divided among the second annular-like areas 142, 142A and 142B whose area sizes may or may not be equal. In an alternative embodiment, the third area 143 is included in the number N if the third area 143 is equipped with array elements and unused.

In another exemplary embodiment, the array is optionally fully populated or sparsely populated by the dedicated transmit elements and the dedicated receive elements. In case of semi-sparsely populated rings, a predetermined Apodization function is applied to weight the detected signals for the purpose of shaping a beam profile.

FIG. 14 also illustrates a certain activation pattern or sequence of the dedicated receive elements in detecting the ultrasound echoes during the receiving operation. During the receive operation, either one of the annular-like areas is activated to detect the ultrasound echoes. The activated annular-like area is optionally a combination of the first annular-like areas 141, 141A and 141B. Alternatively, a combination of the second annular-like area 142, 142A and 142B is activated to detect the ultrasound echoes. In either case, the selected annular-like receive areas are neither dynamically activated nor steered. Thus, the steered. Thus, the annular-like receive areas substantially maintain their spatial relation of the dedicated receive elements.

On the other hand, the third area or Spot of Arago 143 is dynamic during the receive operation. For example, the size of Spot of Arago 143 dynamically changes from a first size 143A to a second size 143B or vice versa during the receive operation. For example, the size of the dynamic Spot of Arago 143 changes due to the first annular-like areas 141, which changes its size by activating or deactivating predetermined portions in a certain sequence during the receive operation. Furthermore, the Spot of Arago 143 dynamically changes with respect to image depth or time in a certain embodiment.

In addition to the above illustrated embodiment, alternative embodiments based upon the embodiment further include an elliptical embodiment and a polygonal embodiment. In the elliptical alternative embodiment, the shared transmit/receive elements, the dedicated transmit elements and the dedicated receive elements are all placed in annular-like elliptical areas including a first annular-like area, a second annular-like area and a sixth annular-like area as described with respect to the above embodiment. Similarly, the third area and the fourth also exist in the elliptical alternative embodiment in a substantially similar manner as described with respect to the above embodiment. By the same token, in the polygonal alternative embodiment, the shared transmit/receive elements, the dedicated transmit elements and the dedicated receive elements are all placed in annular-like polygonal areas including a first annular-like area, a second annular-like area and a sixth annular-like area as described with respect to the above embodiment. Similarly, the third area and the fourth areas also exist in the polygonal alternative embodiment in a substantially similar manner as described with respect to the above embodiment. Although the alternative embodiments are not illustrated in drawings, the alternative embodiments are disclosed by the illustrated embodiment in combination with the above description. The operation of these alternative embodiments is also substantially similar to the above described embodiment.

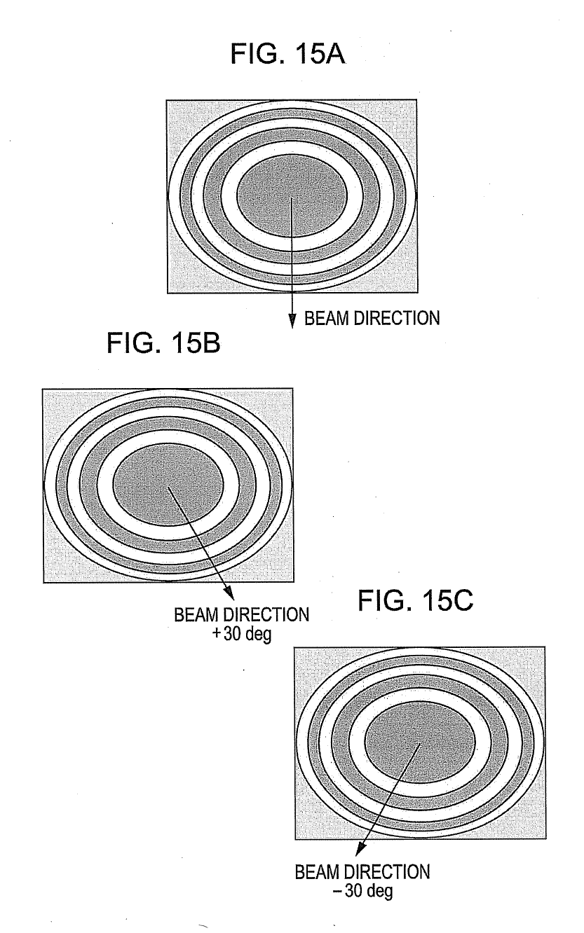

In addition to the above described operations of the embodiments, there are other operations that can be applied to the embodiments of the array in an independent or combined manner. Now referring to FIGS. 15A, 15B and 15C, a spatial compounding aperture technique is illustrated using an embodiment having the array in an elliptical arrangement. In general, the detailed description of the array including the elliptical annular-like areas of the diagram in FIG. 15A is substantially similar to that for the second embodiment as described with respect to the second embodiment as illustrated in FIG. 2. Since the spatial compounding aperture technique is optionally applicable to other embodiments, the technique will be described in its general operational manner. The spatial compounding aperture technique as illustrated in FIGS. 15A, 15B and 15C is merely exemplary and does not limit any aspect of the spatial compounding aperture technique as applied to this or other embodiments.

FIG. 15A illustrates a predetermined beam direction with respect to its transmit aperture during a transmit operation. The substantially same transmit operation is repeated for a predetermined number of times. FIG. 15B illustrates the beam direction with respect to its receive aperture during a first receive operation. The beam direction is steered to +30 degrees in the counter clockwise direction with respect to the transmit beam from the first transmit firing. By the same token, FIG. 15C illustrates the beam direction with respect to its receive aperture during a second receive operation. The beam direction is steered to −30 degrees in the clockwise direction with respect to the transmit beam from the second transmit firing.

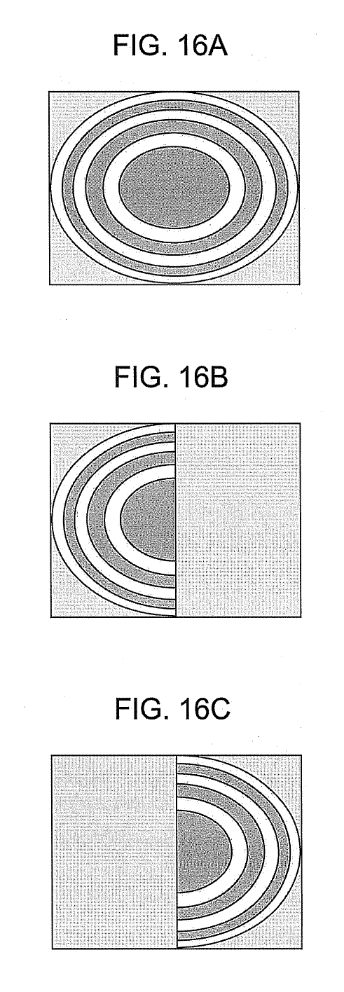

Now referring to FIGS. 16A, 16B and 16C, a synthetic aperture technique is illustrated using an embodiment having the array in an elliptical arrangement. In general, the detailed description of the array including the elliptical annular-like areas of the diagram in FIG. 16A is substantially similar to that for the second embodiment as described with respect to the second embodiment as illustrated in FIG. 2. Since the synthetic aperture technique is optionally applicable to other embodiments, the technique will be described in its general operational manner. The synthetic aperture technique as illustrated in FIGS. 16A, 16B and 16C is merely exemplary and does not limit any aspect of the synthetic aspect of the synthetic aperture technique as applied to this or other embodiments.

FIG. 16A illustrates a transmit aperture during a transmit operation. The substantially same transmit operation is repeated for a predetermined number of times. FIG. 16B illustrates the receive aperture during a first receive operation. A left half of the receiving elements are used to detect echoes with respect to the transmit beam from the first transmit firing. By the same token, FIG. 16C illustrates the receive aperture during a second receive operation. A right half of the receiving elements are used to detect echoes with respect to the transmit beam from the second transmit firing.

As already described, the activation pattern of the transducer elements is not limited to a particular sequence. Although another example of the synthetic aperture techniques is not illustrated in a drawing, the example involves the standard annular-like areas with varied activation patterns both on the transmission and reception as below:
a. On the first transmit, a first half of the transmit elements (t1) is activated to transmit ultrasound pulses while a first half of the receive elements (r1) is activated to receive echoes.
b. On the second transmit, the same first half of the transmit elements (t1) is activated to transmit ultrasound pulses while a second half of the receive elements (r2) is activated to receive echoes.
c. On the third transmit, a second half of the transmit elements (t2) is activated to transmit ultrasound pulses while the first half of the receive elements (r1) is activated to receive echoes.
d. On the fourth transmit, the second half of the transmit elements (t2) is activated to transmit ultrasound pulses and the second half of the receive elements (r2) is activated to receive echoes.

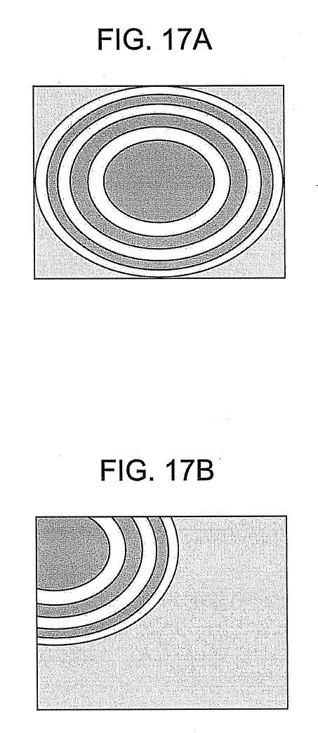

Now referring to FIGS. 17A and 17B, an asymmetric aperture technique is illustrated using an embodiment having the array in an elliptical arrangement. In general, the detailed description of the array including the elliptical annular-like areas of the diagram diagram in FIG. 17A is substantially similar to that for the second embodiment as described with respect to the second embodiment as illustrated in FIG. 2. Since the asymmetric aperture technique is optionally applicable to other embodiments, the technique will be described in its general operational manner. The asymmetric aperture technique as illustrated in FIG. 17B is merely exemplary and does not limit any aspect of the asymmetric aperture technique as applied to this or other embodiments.

FIG. 17A illustrates one example of symmetric apertures when the beam is not steered and centered using an embodiment having the array in an elliptical arrangement. In contrast, FIG. 17B illustrates one example of asymmetric apertures for a larger field of view in virtual apex mode using an embodiment having the array in an elliptical arrangement. In this case, the beam origin intersects the array at different places based on the beam steering angle in 3D acoustic space. In addition, FIG. 17B also illustrates one example of asymmetric apertures during both transmit and receive operations. As illustrated, when the beam is off center and steered to the side, an aperture falls off the edge of the array and results in creating an asymmetric aperture.

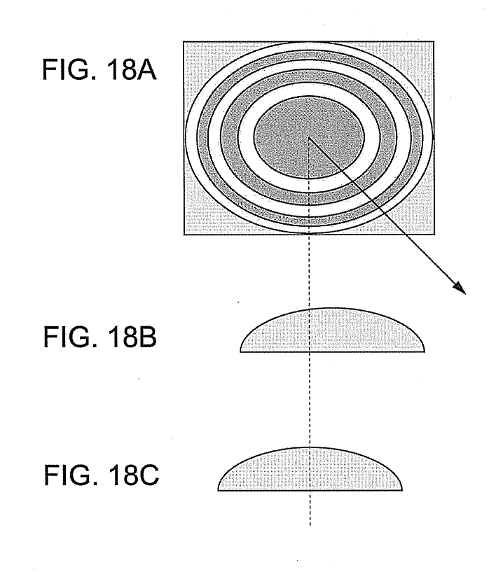

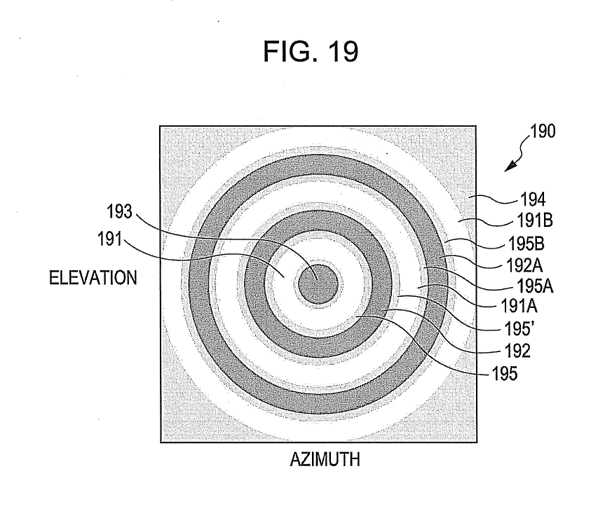

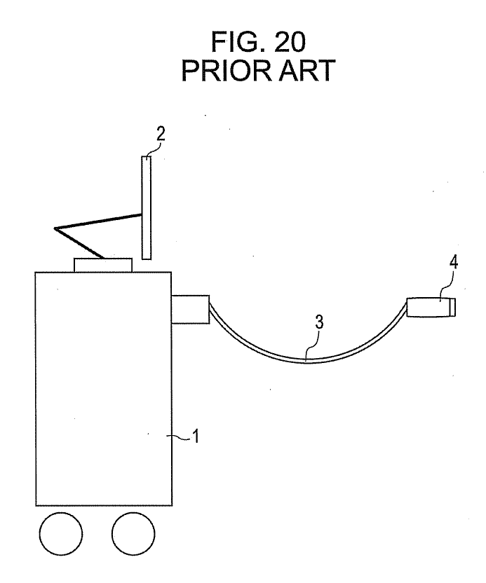

Now referring to FIGS. 18A, 18B and 18C, another example of the asymmetric aperture technique is illustrated using an embodiment having the array in an elliptical arrangement. In general, the detailed description of the array including the elliptical annular-like areas of the diagram in FIG. 18A is substantially similar to that for the second embodiment as described with respect to the second embodiment as illustrated in FIG. 2. Since the asymmetric aperture technique is optionally applicable to other embodiments, the technique will be described in its general operational manner. The asymmetric aperture technique as illustrated in FIGS. 18A, 18B and 18C is merely exemplary and does not limit any aspect of the asymmetric aperture technique as applied to this or other embodiments.

FIG. 18A illustrates one example of symmetric apertures when the beam is steered as indicated by an arrow using an embodiment having the array in an elliptical arrangement. In addition, the center of the elliptical arrangement or beam origin is indicated by a dotted line that is extended to FIGS. 18B and 18C. In the near field, when the beam is steered, the aperture side closer to the focus location may become larger. In general, as the echoes are received from deeper portions of acoustic space, the apodization function becomes centered on the beam origin as illustrated in FIGS. 18B and 18C.

FIG. 18B illustrates one example of asymmetric apertures using an embodiment having the array in an elliptical arrangement at a first depth in acoustic space. At this depth, an aperture falls off the center of the beam origin as indicated by the dotted line and results in creating an asymmetric aperture after apodization weighting is applied to each element. In contrast, FIG. 18C illustrates at a second depth in acoustic space, an aperture falls more on the center of the beam origin as indicated by the dotted line and results in creating a more symmetric aperture after apodization weighting is applied to each element. Consequently, as the depth changes, the effect is to skew the symmetry of the effective aperture after apodization weighting is applied to each element.

FIG. 19 is a diagram illustrating a ninth embodiment having multiple non-overlapping annular-like areas according to the current invention. In general, although the embodiment is similar to the seventh embodiment as illustrated in FIG. 7, it lacks a Spot of Arago. In general, the embodiment is a two-dimensional array 190 of transducer elements that includes dedicated transmit elements that perform only transmit functions and dedicated receive elements that perform only receive functions. That is, the embodiment according to the current invention excludes any shared transmit/receive elements that perform both transmit and receive functions within the same element. The dedicated transmit elements and the dedicated receive elements are placed in a certain predetermined spatial arrangement as indicated by different shades of color in the diagram.

The dedicated transmit elements and the dedicated receive elements are both placed in annular-like circular areas including a first annular-like area 191 and a second annular-like area 192. As indicated by different shades, the first annular-like area 191 exclusively includes either one of dedicated transmit elements or dedicated receive elements while the second annular-like area 192 area exclusively includes the other one of the dedicated transmit elements and the dedicated receive elements. In other words, the first annular-like area 191 and the second annular-like area 192 alternate the dedicated transmit elements and the dedicated receive elements in their respective annular-like circular areas. For example, if the first annular-like area 191 exclusively includes the dedicated transmit elements, the second annular-like area 192 exclusively includes the dedicated receive elements. Although the second annular-like area 192 is not immediately juxtaposed around the first annular-like area 191, the second annular-like area 192 has a substantially concentric center with the first annular-like area 191.

In the embodiment of the array in the probe, there is an optional annular-like area 195 between the first annular-like area 191 and the second annular-like area 192. The optional annular-like area 195 is optionally populated with either one of the dedicated transmit elements or the dedicated receive elements, and these elements may be also optionally used or disabled. Alternatively, the optional annular-like area 195 is optionally populated with neither one of the dedicated transmit elements or the dedicated receive elements. Furthermore, an additional optional annular-like area 195′ surrounds the second annular-like area 192, and the additional optional annular-like area 195′ may be implemented in a similar manner as the optional annular-like area 195.

As illustrated in the diagram, the first annular-like area 191 and the second annular-like area 192 are optionally repeated over a predetermined transducer surface of the two-dimensional array 190. As indicated by the shaded circular rings in the diagram, the additionally repeated annular-like areas 191A, 192A and 191B also exclusively have an alternate one of the dedicated transmit elements and the dedicated receive elements. In the illustrated embodiment, as the second annular-like area 192 is larger than the first annular-like area 191 and is not immediately juxtaposed around the first annular-like area 191, the additionally repeated annular-like areas 191A, 192A and 191B also have substantially the same spatial relationship among them. By the same token, the additionally repeated annular-like areas 191A, 192A and 191B are interlaced by optional annular-like areas 195A and 195B. The term, “annular-like area” is intended to have the same meaning as already described with respect to FIG. 1 in the in the current patent application.

Still referring to FIG. 19, the exemplary embodiment additionally includes a third area 193 and a fourth area 194. The third area 193 is a circle and is located inside the first annular-like area 191 and at least over the concentric center. The third area 193 is optionally juxtaposed to the first annular-like area 191 or alternatively contained in the first annular-like area 191 with a gap between the third area 193 and the first annular-like area 191. In this embodiment, the third area 193 contains the same elements as the second annular-like area 192.

In contrast, the fourth area 194 is located outside the largest annular-like area 191B on the two-dimensional array surface. The fourth area 194 is optionally disabled or devoid of any functional transducer element. Alternatively, the fourth area 194 is optionally populated by the dedicated transmit elements for maximum power and/or the dedicated receive elements for maximum sensitivity.

In another exemplary embodiment, the array is optionally fully populated or sparsely populated by the dedicated transmit elements and the dedicated receive elements. In case of semi-sparsely populated rings, a predetermined Apodization function is applied to weight the detected signals for the purpose of shaping a beam profile.

According to any and or all of the above described embodiments, at least the following advantages are substantially achieved. The use of dedicated receive elements, dedicated transmit elements and or Spot of Arago lowers implementation costs of the related electronics.

Furthermore, with respect to the transmit and or receive operations, beam width is advantageously optimized particularly in the near field, and the optimized beam width results in better resolution of an image. Sidelobes are also advantageously optimized particularly in the near field, and the optimized sidelobes result in reduced noise in an image.

While certain embodiments have been described above, these embodiments have been presented by way of example only and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope of the inventions.

1. An array in an ultrasound probe, comprising:
at least one first annular-like area exclusively including either one of dedicated transmit elements or dedicated receive elements; and
at least one second annular-like area being immediately juxtaposed around said first annular-like area and having a substantially concentric center with said first annular-like area, said second annular-like area exclusively including the other one of said dedicated transmit elements and said dedicated receive elements.

2. (canceled)

3. The array according to claim 1 further comprising a third area located inside said first annular-like area and at least over the concentric center, said third area being operationally disabled and acting as Spot of Arago.

4. The array according to claim 3 wherein a size of said third area is dynamic with respect to a combination of depth and steering angle when said third area exclusively includes said dedicated receive elements.

5. The array according to claim 1 further comprising a third area located inside said first annular-like area and at least over the concentric center, said third area acting as Spot of Arago and including neither of said dedicated transmit elements and said dedicated receive elements.

6. The array according to claim 1 further comprising a fourth area located outside said second annular area exclusively including a combination of said dedicated transmit elements and said dedicated receive elements.

7. The array according to claim 6 wherein said fourth area is disabled.

8. (canceled)

9. (canceled)

10. (canceled)

11. The array according to claim 1 wherein said first annular-like area is smaller than said second annular-like area in area.

12. The array according to claim 1 wherein said first annular-like area is larger than said second annular-like area in area.

13. (canceled)

14. (canceled)

15. The array according to claim 1 wherein at least one of said first annular-like area and said second annular-like area is substantially ecliptic.

16. The array according to claim 1 wherein at least one of said first annular-like area and said second annular-like area is polygonal.

17. (canceled)

18. (canceled)

19. The array according to claim 18 wherein said Apodization function changes with depth.

20. The array according to claim 1 wherein one of said first annular-like area and said second annular-like area is dynamic with respect to a steering angle when said one of said first annular-like area and said second annular-like area exclusively includes said dedicated receive elements.

21. The array according to claim 20 wherein said steering angle is changed to a predetermined angle for each of receive operations for spatial compounding apertures.

22. The array according to claim 20 wherein said dedicated receive elements have an asymmetric apertures.

23. The array according to claim 1 wherein one of said first annular-like area and said second annular-like area exclusively includes said dedicated receive elements, a predetermined portion of said dedicated receive elements being activated for each of receive operations for generating synthetic apertures.

24. The array according to claim 20 further comprising a third area located inside said first annular-like area and at least over the concentric center, said third area being Spot of Arago.

25. The array according to claim 20 further comprising a third area located inside said first annular-like area and at least over the concentric center, said third area exclusively including said dedicated receive elements, said third area being dynamic Spot of Arago with respect to depth.

26. The array according to claim 20 further comprising a third area located inside said first annular-like area and at least over the concentric center, said third area exclusively including the other one of said dedicated transmit elements and said dedicated receive elements.

27. An array in an ultrasound probe, comprising:
at least one first annular-like area exclusively including either one of dedicated transmit elements or dedicated receive elements;
at least one second annular-like area being substantially around said first annular-like area and having a substantially concentric center with said first annular-like area, said second annular-like area exclusively including the other one of said dedicated transmit elements and said dedicated receive elements; and
a third area located inside said first annular-like area and at least over the concentric center, said third area being Spot of Arago.

28. The array according to claim 27 wherein said third area being operationally disabled.

29. The array according to claim 27 wherein a size of said third area is dynamic with respect to depth when said third area exclusively includes said dedicated receive elements.

30. The array according to claim 27 wherein said third area acting as Spot of Arago and including neither of said dedicated transmit elements and said dedicated receive elements.

31. The array according to claim 27 further comprising a fourth area located outside said second annular area exclusively including either one of said dedicated transmit elements and said dedicated receive elements.

32. The array according to claim 31 wherein said fourth area is disabled.

33. The array according to claim 27 further comprising a fourth area located outside said second annular area including neither one of said dedicated transmit elements and said dedicated receive elements.

34. The array according to claim 27 wherein said first annular-like area and said second annular-like area are substantially equal in area.

35. The array according to claim 27 wherein said first annular-like area is smaller than said second annular-like area in area.

36. The array according to claim 27 wherein said first annular-like area is larger than said second annular-like area in area.

37. The array according to claim 27 wherein at least one of said first annular-like area and said second annular-like area is substantially circular.

38. The array according to claim 27 wherein said first annular-like area and said second annular-like area are both circular and each have a radius rₙ as follows:
$${r}_{n} = \sqrt{n λ f + \frac{{n}^{2} {λ}^{2}}{4}}$$
where n is an integer while λ is a wavelength of ultrasound waves the array is meant to focus and a focus f is the distance from the center of the array to the focus.

39. The array according to claim 27 herein at least one of said first annular-like area and said second annular-like area is substantially elliptical.

40. The array according to claim 27 wherein at least one of said first annular-like area and said second annular-like area is polygonal.

41. The array according to claim 27 wherein at least one of said first annular-like area and said second annular-like area is semi-sparsely populated with array elements.

42. The array according to claim 41 wherein said semi-sparsely populated array elements are approximated by a predetermined Apodization function.

43. The array according to claim 42 wherein said Apodization function changes with depth.

44. The array according to claim 2 wherein one of said first annular-like area and said second annular-like area is dynamic with respect to a steering angle when said one of said first annular-like area and said second annular-like area exclusively includes said dedicated receive elements.

45. The array according to claim 44 wherein said steering angle is changed to a predetermined angle for each of receive operations for spatial compounding apertures.

46. The array according to claim 44 wherein said dedicated receive elements have an asymmetric apertures.

47. The array according to claim 27 wherein one of said first annular-like area and said second annular-like area exclusively includes said dedicated receive elements, a predetermined portion of said dedicated receive elements being activated for each of receive operations for generating synthetic apertures.

48. The array according to claim 44 wherein said third area exclusively includes said dedicated receive elements, said third area being dynamic with respect to depth.

49. The array according to claim 27 wherein said second annular-like area being immediately juxtaposed around said first annular-like area.

50. The array according to claim 27 further comprising a fifth area located between said second annular-like area and said first annular-like area and exclusively including either one of said dedicated transmit elements and said dedicated receive elements.

51. The array according to claim 50 wherein said fifth area is disabled.

52. The array according to claim 27 further comprising a fifth area located between said second annular area and said first annular-like area and including neither one of said dedicated transmit elements and said dedicated receive elements.

53. The array according to claim 31 further comprising a fifth area located between said second annular-like area and said first annular-like area and exclusively including either one of said dedicated transmit elements and said dedicated receive elements.

54. The array according to claim 53 wherein said fifth area is disabled.

55. The array according to claim 33 further comprising a fifth area located between said second annular area and said first annular-like area and including neither one of said dedicated transmit elements and said dedicated receive elements.

56. The array according to claim 27 further comprising a sixth annular-like area located between said second annular-like area and said first annular-like area and including both of said dedicated transmit elements and said dedicated receive elements.

57. The array according to claim 56 wherein said sixth annular-like area is juxtaposed to said second annular-like area and said first annular-like area.

58. An array in an ultrasound probe, comprising:
at least one first annular-like area exclusively including either one of dedicated transmit elements or dedicated receive elements;
at least one second annular-like area being substantially around said first annular-like area and having a substantially concentric center with said first annular-like area, said second annular-like area exclusively including the other one of said dedicated transmit elements and said dedicated receive elements; and
a third area located inside said first annular-like area and at least over the concentric center, said third area exclusively including the other one of said dedicated transmit elements and said dedicated receive elements.
